(19) United States
(12) Patent Application Publication
(10) Pub. No.: US 2019/0224086 A1
(43) Pub. Date: Jul. 25, 2019

(54) MICROCAPSULE COMPOSITIONS WITH IMPROVED DEPOSITION

(71) Applicant: INTERNATIONAL FLAVORS & FRAGRANCES INC., New York, NY (US)

(72) Inventors: Laurence SOUBIRAN, Le Mesnil Le Roi (FR); Clémentine MARTEAU-ROUSSY, Vincennes (FR); Michele WHITE, Oceanport, NJ (US); Christopher Thomas LAVALLEE, Morganville, NJ (US); Yabin LEI, Holmdel, NJ (US); Li XU, Edison, NJ (US)

(21) Appl. No.: 16/371,393

(22) Filed: Apr. 1, 2019

Related U.S. Application Data
(63) Continuation-in-part of application No. 15/709,612, filed on Sep. 20, 2017.
Application No. 15/709,612 is a continuation-in-part of application No. PCT/US2017/051916, filed on Sep. 15, 2017.
Application No. PCT/US2017/051916 is a continuation-in-part of application No. 15/047,364, filed on Feb. 18, 2016, now Pat. No. 10,085,925.
(60) Provisional application No. 62/395,586, filed on Sep. 16, 2016.
(60) Provisional application No. 62/272,740, filed on Dec. 30, 2015.

(51) Int. Cl. A61K 8/11; A61Q 5/02; A61K 8/81; A61K 8/73; A61K 8/87; A61K 8/92; A61Q 13/00; A61Q 19/10

(52) U.S. Cl. CPC A61K 8/11; A61Q 5/02; A61K 8/8117; A61K 8/731; A61K 8/8111; A61K 2800/654; A61K 8/922; A61Q 13/00; A61Q 19/10; A61K 2800/56; A61K 8/87

(57) ABSTRACT

A microcapsule composition useful in delivering an active material contains an oil core and a microcapsule wall encapsulating the oil core. The microcapsule has a zeta potential of 10 mV or greater, the microcapsule wall is formed of an encapsulating polymer, and the oil core contains an active material. Also disclosed are consumer products containing such a microcapsule.

### CROSS-REFERENCE TO RELATED APPLICATIONS

This application is a continuation-in-part application of U.S. application Ser. No. 15/709,612, filed Sep. 20, 2017, which is a continuation-in-part application of International Application No. PCT/US2017/051916, filed Sep. 15, 2017, which claims priority to U.S. Application Ser. No. 62/395,586, filed Sep. 16, 2016. This application is also a continuation-in-part application of U.S. application Ser. No. 15/047,364, filed on Feb. 18, 2016, now issued as U.S. Pat. No. 10,085,925 which claims priority to U.S. Application Ser. No. 62/272,740, filed on Dec. 30, 2015. The contents of the above-mentioned applications are incorporated herein by reference in their entireties.

### BACKGROUND

Nano- or micro-encapsulation is used in a variety of different applications where there is a need to deliver, apply, or release an active material including a fragrance, flavor, and malodor counteraction agent to a target area in a time-delayed or controlled manner. Various techniques for preparing capsules are known in the art and are used, depending on the contents to be encapsulated, the environment in which the capsules should retain their integrity and the desired release mechanism.

Interfacial polycondensation is a known technique for preparing capsules and versatile capsule wall materials including polyureas and polyurethanes (see WO 2011/154893 and WO 2012/107323). Such wall materials are produced by having a first phase which is water-immiscible and includes a polyfunctional isocyanate, i.e., a polyisocyanate having two or more isocyanate groups, and a second aqueous phase which includes (i) a polyfunctional alcohol (i.e., a polyol) having two or more —OH groups for obtaining a polyurethane capsule wall, or (ii) a polyfunctional amine (i.e., a polyamine) having two or more —NH₂ and/or —NH groups for obtaining a polyurea capsule wall.

If the active material to be encapsulated is hydrophobic, it will be included in the water-immiscible phase, thereafter the two phases are mixed by high shear mixing to form an oil-in-water emulsion. In this emulsion, the polycondensation reaction will take place. Thus, the small droplets of the water-immiscible phase will be surrounded by the capsule wall formed by polycondensation of the isocyanate and the polyalcohol or polyamine as starting materials. Conversely, if the material to be encapsulated is hydrophilic, it will be included in the aqueous phase and the mixture of the two phases converted into a water-in-oil emulsion. The polycondensation reaction will then form capsule walls surrounding the droplets of water-miscible phase. Suitable emulsifiers are often utilized to aid in the preparation and stabilization of the emulsion.

Suitable raw materials and processes for preparing capsules by polycondensation are described in patents such as U.S. Pat. Nos. 4,640,709 and 6,133,197. WO 2011/154893 discloses a process for the preparation of capsules, which includes mixing at least one aliphatic polyisocyanate and of at least one aromatic polyisocyanate, wherein the molar ratio between the two polyisocyanates is between 75:25 and 20:80.

WO 2013/000587 discloses a process for the preparation of polyurea capsules, which includes dissolving at least one polyisocyanate having at least two isocyanate functional groups, in a perfume to form a solution; adding to the solution an aqueous solution of an emulsifier or of a colloidal stabilizer; and adding to the mixture to 3,5-diamino-1,2,4-triazole to form a polyurea wall.

U.S. Pat. No. 5,304,448 describes an encapsulated toner composition using reaction of amino acids and polyisocyanates.

Known polyurea or polyurethane capsules face various issues, e.g., low olfactory intensity, low stability, and high toxicity. Their deposition to target surfaces is also problematic.

There is a need to develop a safe, stable, and high efficient capsules for use in laundry, washing, cleaning, surface care and personal and skin care. For such applications quicker and easier release and/or less mechanical strength are often desirable. Also, it would be desirable to more precisely influence the capsule wall permeability and other capsule wall properties to achieve the desired release profile and consumer benefits.

### SUMMARY OF THE INVENTION

This invention is based on the discovery that certain capsule compositions possess unexpected desirable properties including high perceived olfactory intensity and improved deposition.

Accordingly, one aspect of this invention relates to microcapsule compositions containing a microcapsule and a copolymer of acrylamidopropyl trimonium chloride and acrylamide as a first deposition aid to facilitate the deposition of the microcapsule onto a hard surface such as skin, hair, fabric, furniture, and floor.

The microcapsule composition contains by weight 0.1 wt % to 90 wt % of the microcapsule and 0.01 wt % to 50 wt % of the copolymer of acrylamidopropyl trimonium chloride and acrylamide.

The microcapsule can have a zeta potential of a zeta potential of 10 mV or greater (e.g., 25 mV or greater, 25 to 200 mV, 40 mV or greater, and 40 mV to 100 mV) and a rupture force of 5 mN or less (e.g., 2 mN or less). The size of the microcapsule is typically in the range of 0.1 micron to 1000 microns with a lower limit of 0.1 micron, 0.2 micron, 0.5 micron, 1 micron, or 2 microns and an upper limit of 1000 microns, 500 microns, 200 microns, 100 microns, 50 microns, 30 microns, or 20 microns (e.g., 0.5 micron to 500 microns, 0.5 micron to 200 microns, 1 micron to 100 microns, and 2 microns to 50 microns).

The microcapsule has an oil core and a microcapsule wall encapsulating the oil core. The oil core can contain a fragrance. The wall is typically formed of an encapsulating polymer, e.g., the reaction product of a multi-functional electrophile and a multi-functional nucleophile.

The multi-functional electrophile contains a polyisocyanate such as a trimer of hexamethylene diisocyanate, a trimer of isophorone diisocyanate, a biuret of hexamethylene diisocyanate, a polyisocyanurate of toluene diisocyanate, a trimethylol propane-adduct of toluene diisocyanate, a trimethylol propane-adduct of xylylene diisocyanate, and combinations thereof.

The multi-functional nucleophile contains a branched polyethyleneimine, which preferably having a molecular weight of 750 Daltons to 500000 Daltons with a lower limit of 750 Daltons, 1000 Daltons, 2000 Daltons, 5000 Daltons, 10000 Daltons or 15000 Daltons and an upper limit of 500000 Daltons, 200000 Daltons, 100000 Daltons, or 50000 Daltons. In some embodiments, the multi-functional nucleophile is a mixture of a branched polyethyleneimine and a polyamine selected from the group consisting of hexamethylenediamine, ethylenediamine, 1,3-diaminopropane, 1,4-diamino-butane, diethylenetriamine, pentaethylenehexamine, bis(3-aminopropyl)amine, bis(hexanethylene)triamine, tris(2-aminoethyl)amine, triethylene-tetramine, N,N′-bis(3-aminopropyl)-1,3-propanediamine, tetraethylenepentamine, penta-ethylenehexamine, chitosan, nisin, gelatin, 1,3-diamino-guanidine, 1,1-dimethylbiguanide, guanidine, arginine, lysine, ornithine, and combinations thereof.

In some embodiments, the microcapsule compositions contain only one deposition aid, i.e., the acrylamidopropyl trimonium chloride and acrylamide copolymer, and are free of another deposition aid. In other embodiments, the microcapsule compositions contain a second deposition aid, e.g., polyquaternium-4, polyquaternium-5, polyquaternium-6, polyquaternium-7, polyquaternium-10, polyquaternium-16, polyquaternium-22, polyquaternium-24, polyquaternium-28, polyquaternium-39, polyquaternium-44, polyquaternium-46, polyquaternium-47, polyquaternium-53, polyquaternium-55, polyquaternium-67, polyquaternium-68, polyquaternium-69, polyquaternium-73, polyquaternium-74, polyquaternium-77, polyquaternium-78, polyquaternium-79, polyquaternium-80, polyquaternium-81, polyquaternium-82, polyquaternium-86, polyquaternium-88, polyquaternium-101, polyvinylamine, polyethyleneimine, polyvinylamine and vinylformamide copolymer, and combinations thereof.

Further, the microcapsule compositions can also contain a capsule formation aid selected from the group consisting of a polyvinyl alcohol, polystyrene sulfonate, carboxymethyl cellulose, naphthalene sulfonate condensate salt, polyvinylpyrrolidone, copolymer of vinyl pyrrolidone and quaternized dimethylaminoethyl methacrylate, and combinations thereof.

It is possible that the microcapsule compositions contain a second, third, fourth, fifth, or sixth delivery system, each of which can be a microcapsule different from each other. The microcapsule compositions can be in the form of either a solid or liquid.

Another aspect of this invention relates to consumer products containing any of the microcapsule compositions described above. Exemplary consumer products include hair care products (e.g., shampoos and hair conditioners), personal care products (e.g., shower gels and bar soaps), fabric care products (e.g., powder or liquid fabric detergents, fabric conditioners, and fabric refreshers), and home care products.

Also within the scope of this invention is a method of preparing a shampoo composition comprising the step of: (i) providing a shampoo base containing 2 wt % to 35 wt % of a surfactant, and (ii) adding into the shampoo base a microcapsule and a copolymer of acrylamidopropyl trimonium chloride and acrylamide to obtain the shampoo composition, wherein the shampoo composition contains by weight (a) 0.001% to 10% of the copolymer of copolymer of acrylamidopropyl trimonium chloride and acrylamide, (b) 0.01% to 10% of the microcapsule, and (c) the microcapsule contains a microcapsule core and a microcapsule wall encapsulating the microcapsule core, the microcapsule core having a fragrance and the microcapsule wall formed of an encapsulating polymer.

The microcapsule can be prepared by the steps of: (iii) preparing an oil phase containing a fragrance and at least one polyisocyanate; (iv) preparing an aqueous phase containing a microcapsule formation aid; (v) emulsifying the oil phase into the aqueous phase to form a fragrance emulsion; (vi) adding at least one cross-linking agent to the fragrance emulsion to form a polyurea capsule slurry; and (e) curing the polyurea capsule slurry. In some embodiments, the polyisocyanate contains a trimethylol propane-adduct of xylylene diisocyanate and is present in a polyisocyanate solution having a flash point of at least 60° C. In other embodiments, the polyisocyanate solution contains a solvent selected from the group consisting of triacetin, triethyl citrate, ethylene glycol diacetate, and combinations thereof.

The details of one or more embodiments of the invention are set forth in the description below. Other features, objects, and advantages of the invention will be apparent from the description and the claims.

### DETAILED DESCRIPTION OF THE INVENTION

It has been found that certain microcapsules have unexpected high performance (e.g., fragrance intensity) in many applications (e.g., hair conditioners and fabric conditioners). These microcapsules have a zeta potential of 10 mV or greater.

The microcapsules are useful in a wide range of consumer applications, e.g., personal hair care products including shampoos and hair conditioners; personal washes such as soaps, body wash, personal cleaners, and sanitizers; fabric care such as fabric refreshers, softeners, and dryer sheets; ironing water; industrial cleaners; liquid and powder detergent; rinse conditioners; fine fragrances; an Eau De Toilette product; a deodorant; a roll-on product; and an aerosol product. The capsule compositions of this invention are also well-suited for use in hydroalcoholic medium such as fine fragrance and for use in leave-on personal care applications. Moreover, the inclusion of a capsule formation aid in the capsule wall-forming reaction provides capsules with excellent storage stability and retention of an encapsulated fragrance.

The microcapsules of this invention are core-shell microcapsules each containing an oil core and a microcapsule wall encapsulating the oil core.

The microcapsules can be positively charged with a zeta potential of at least 10 mV (e.g., 25 mV or greater, 40 mV or greater, 25 mV to 200 mV, and 40 mV to 100 mV). Not to be bound by theory, the positively charged microcapsules have a strong affinity to specific animate and inanimate surfaces (e.g., hair and fabric), and also are unexpectedly stable in certain consumer product bases such as hair conditioners, shampoos, shower gels, and fabric conditioners.

The microcapsules of this invention can be prepared by reacting (e.g., via an interfacial polymerization) a polyfunctional nucleophile and a polyfunctional electrophile in the presence of a capsule formation aid (e.g., a dispersant) and/or a catalyst (e.g., a base) so that an active material is encapsulated in the oil core by the microcapsule wall. The oil core optionally contains a core modifier. The microcapsule wall is formed of an encapsulating polymer that is the reaction product of a polyfunctional nucleophile and a polyfunctional electrophile.

Preferably, the microcapsule has a microcapsule wall formed of an encapsulating polymer that is a reaction product of a branched polyethyleneimine (a polyfunctional nucleophile) and an aromatic/aliphatic polyisocyanate (a polyfunctional electrophile).

### Polyfunctional Nucleophile

The polyfunctional nucleophile is a branched polyethyleneimine or a mixture containing a branched polyethyleneimine and a polyfunctional amine/alcohol. In a preferred embodiment, the polyfunctional nucleophile is a branched polyethyleneimine.

Suitable branched polyethyleneimines each have a molecular weight of 200 Da to 1000000 Da (e.g., 300 Da to 500000 Da, 500 Da to 200000 Da, 750 Da to 100000 Da, and 750 Da to 50000 Da). They have a main chain and one or more side chains attached to the main chain. The main chain has 2 to 25000 (e.g., 3 to 10000, 5 to 5000, and 5 to 500) repeat ethylene amine (—CH₂CH₂NH—) units. The side chains each have one or more ethylene amine terminals (—CH₂CH₂NH₂). The representative structure of the branched polyethyleneimine is shown below:

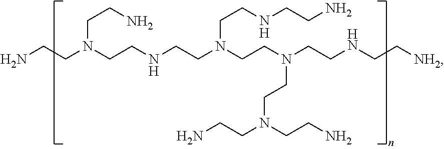

in which n is 1 to 5000 (e.g., 1 to 2000, 1 to 1000, and 1 to 100).

Other suitable polyfunctional nucleophiles include polyfunctional amines, (e.g., polyamines) and polyfunctional alcohols (e.g., polyols).

Polyfunctional amines are those having at least a primary/secondary amine group (—NH₂ and —NH—) and one or more additional functional groups such as a primary/secondary amine and hydroxyl group (—OH). Exemplary polyfunctional amines include hexamethylenediamine, hexaethylenediamine, ethylenediamine, 1,3-diaminopropane, 1,4-diamino-butane, diethylenetriamine, pentaethylenehexamine, bis(3-aminopropyl)amine, bis(hexanethylene)triamine, tris(2-aminoethyl)amine, triethylene-tetramine, N,N′-bis(3-aminopropyl)-1,3-propanediamine, tetraethylenepentamine, amino-2-methyl-1-propanol, a second branched polyethylenimine, chitosan, 1,3-diamino-guanidine, 1,1-dimethylbiguanide, and guanidine. Suitable amino acids/peptides include arginine, lysine, histidine, ornithine, nisin, and gelatin.

Preferred polyfunctional amines are polyamines containing two or more amine groups such as —NH₂ and —R*NH, R* being substituted and unsubstituted C₁-C₂₀ alkyl, C₁-C₂₀ heteroalkyl, C₁-C₂₀ cycloalkyl, 3- to 8-membered heterocycloalkyl, aryl, and heteroaryl.

Two classes of such polyamines include polyalkylene polyamines having the following structures:

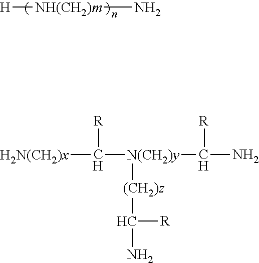

in which R is hydrogen or —CH₃; and each of m, n, x, y, and z, independently, is an integer from 0-2000 (e.g., 1-1000, 1-100, 1-10, and 1-5). Examples include ethylene diamine, 1,3-diaminepropane, diethylene triamine, triethylene tetramine, 1,4-diaminobutane, hexanethylene diamine, hexamethylene diamine, pentaethylenehexamine, and the like.

Another class of polyamines are polyalkylene polyamines of the type:

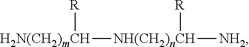

where R equals hydrogen or —CH₃, m is 1-5 and n is 1-5, e.g., diethylene triamine, triethylene tetraamine and the like. Exemplary amines of this type also include diethylenetriamine, bis(3-aminopropyl)amine, bis(hexanethylene)triamine.

Another class of amines that can be used in the invention is polyetheramines. They contain primary amino groups attached to the end of a polyether backbone. The polyether backbone is normally based on either propylene oxide (PO), ethylene oxide (EO), or mixed PO/EO. The ether amine can be monoamine, diamine, or triamine, based on this core structure. An example is:

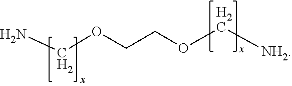

Additional examples include 2,2′-ethylenedioxy)bis(ethylamine) and 4,7,10-trioxa-1,13-tridecanediamine.

Other suitable amines include, but are not limited to, tris(2-aminoethyl)amine, triethylenetetramine, N,N′-bis(3-aminopropyl)-1,3-propanediamine, tetraethylene pentamine, 1,2-diaminopropane, N,N,N′,N′-tetrakis(2-hydroxyethyl)ethylene diamine, N,N,N′,N′-tetrakis(2-hydroxypropyl)ethylene diamine, branched polyethylenimine, 2,4-diamino-6-hydroxypyrimidine and 2,4,6-triaminopyrimidine.

Amphoteric amines, i.e., amines that can react as an acid and a base, are another class of amines of use in this invention. Examples include proteins and amino acids such as gelatin, L-lysine, D-lysine, L-arginine, D-arginine, L-lysine monohydrochloride, D-lysine monohydro-chloride, L-arginine monohydrochloride, D-arginine monohydrochloride, L-ornithine monohydrochloride, D-ornithine monohydrochloride, and mixtures thereof.

Guanidine amines and guanidine salts are yet another class of multi-functional amines of use in this invention. Exemplary guanidine amines and guanidine salts include, but are not limited to, 1,3-diaminoguanidine monohydrochloride, 1,1-dimethylbiguanide hydrochloride, guanidine carbonate and guanidine hydrochloride.

Commercially available examples of amines include polyoxyalkylene polyamines sold under the trademarks JEFFAMINE® EDR-148 (where x=2), JEFFAMINE® EDR-176 (where x=3) (from Huntsman). Other polyamines are sold under the trademarks JEFFAMINE® ED Series and JEFFAMINE® triamines; polyethylenimines from BASF (Ludwigshafen, Germany), e.g., Lupasol FG, Lupasol G20 waterfree, Lupasol PR 8515, Lupasol WF, Lupasol FC, Lupasol G20, Lupasol G35, Lupasol G100, Lupasol G500, Lupasol HF, Lupasol PS, Lupasol HEO 1, Lupasol PN50, Lupasol PN60, Lupasol P0100 and Lupasol SK. Other commercially available polyethylenimines are sold under the trademarks EPOMIN® P-1000, EPOMIN® P-1050, EPOMIN® RP18W and EPOMIN® PP-061 from Nippon Shokubai (New York, N.Y.). Polyvinylamines such as those sold under the trademark LUPAMIN® by BASF can also be used. A wide range of polyetheramines may be selected by those skilled in the art. In certain embodiments, the polyfunctional nucleophile is hexamethylene diamine, polyetheramine or a mixture thereof.

Polyfunctional alcohols are those having two or more hydroxyl groups. Non-limiting examples are pentaerythritol, glucose, 2-aminoethanol, dipentaerythritol, glycerol, polyglycerol, ethylene glycol, hexylene glycol, polyethylene glycol, trimethylolpropane, neopentyl glycol, sorbitol, erythritol, threitol, arabitol, xylitol, ribitol, mannitol, galactitol, fucitol, iditol, inositol, volemitol, isomalt, maltitol, lactitol, maltotriitol, maltotetraitol, polyglycitol, and combinations thereof. More suitable polyfunctional alcohols are described in WO 2015/023961.

The polyfunctional nucleophile as used in this invention can be a single compound (e.g., a branched polyethyleneimine) or a mixture of a branched polyethyleneimine with one or more polyfunctional amines/alcohols.

The range of polyfunctional nucleophile content can vary from 0.05% to 5% (e.g., 0.1% to 3%, 0.1% to 2%, 0.25% to 2%, and 0.25% to 1%) by weight of the microcapsule composition.

In one embodiment, the polyfunctional nucleophile is added to the polymerization reaction at a temperature of 0° C. to 55° C. (e.g., 10° C. to 50° C., 15° C. to 45° C., 20° C. to 40° C., and 22° C. to 35° C.)

### Polyfunctional Electrophiles

The polyfunctional electrophile has at least two electrophilic functional groups reactive towards the branched polyethyleneimine, the polyfunctional amine, or the polyfunctional alcohol to form a network of the encapsulating polymer. Examples of the electrophilic group include formyl, keto, carboxyl, an isocyanate group, a carboxylate ester group, an acyl halide group, an amide group, a carboxylic anhydride group, an alkyl halide group, an epoxide group, an aziridine group, an oxetane group, an azetidine group, a sulfonyl halide group, a chlorophosphate group, an α,β-unsaturated carbonyl group, an α,β-unsaturated nitrile group, a trifluoromethanesulfonate group, a p-toluenesulfonate group, and an α,β-unsaturated methane sulfonyl group.

Suitable polyfunctional electrophiles include glutaric dialdehyde, succinic dialdehyde, and glyoxal; as well as compounds such as glyoxyl trimer and paraformaldehyde, bis(dimethyl) acetal, bis(diethyl) acetal, polymeric dialdehydes, such as oxidized starch. Preferably the cross-linking agent is a low molecular weight, difunctional aldehyde, such as glyoxal, 1,3-propane dialdehyde, 1,4-butane dialdehyde, 1,5-pentane dialdehyde, or 1,6-hexane.

Other non-limiting polyfunctional electrophiles are polyfunctional isocyanates (i.e., polyisocyanate), each of which contains two or more isocyanate (—NCO) groups. These polyisocyanate can be aromatic, aliphatic, linear, branched, or cyclic. In certain embodiments, the polyisocyanate contains, on average, 2 to 4 isocyanate groups. In particular embodiments, the polyisocyanate contains at least three isocyanate functional groups. In certain embodiments, the polyisocyanate is water insoluble.

In particular embodiments, the polyisocyanate used in this invention is an aromatic polyisocyanate. Desirably, the aromatic polyisocyanate includes a phenyl, tolyl, xylyl, naphthyl or diphenyl moiety as the aromatic component. In certain embodiments, the aromatic polyisocyanate is a polyisocyanurate of toluene diisocyanate, a trimethylol propane-adduct of toluene diisocyanate or a trimethylol propane-adduct of xylylene diisocyanate.

One class of suitable aromatic polyisocyanates are those having the generic structure shown below, and its structural isomers

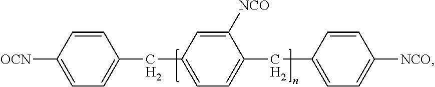

wherein n can vary from zero to a desired number (e.g., 0-50, 0-20, 0-10, and 0-6) depending on the type of polyamine or polyol used. Preferably, the number of n is limited to less than 6. The starting polyisocyanate may also be a mixture of polyisocyanates where the value of n can vary from 0 to 6. In the case where the starting polyisocyanate is a mixture of various polyisocyanates, the average value of n preferably falls in between 0.5 and 1.5. Commercially-available polyisocyanates include those sold under the trademarks LUPRANATE® M20 (BASF; PMDI containing isocyanate group “NCO” at 31.5 wt %), where the average n is 0.7; PAPI® (Dow Chemical; PMDI having an average molecular weight of 340 and containing NCO at 31.4 wt %) where the average n is 0.7; MONDUR MR® (Bayer; PMDI containing NCO at 31 wt % or greater) where the average n is 0.8; MONDUR MR® Light (Bayer; PMDI containing NCO at 31.8 wt %) where the average n is 0.8; MONDUR® 489 (Bayer; PMDI containing NCO at 30-31.4 wt %) where the average n is 1; poly[(phenylisocyanate)-co-formaldehyde] (Aldrich Chemical, Milwaukee, Wis.), and other isocyanate monomers sold under the trademarks DESMODUR® N3200 (Bayer; poly(hexamethylene diisocyanate), and TAKENATE® D110-N (Mitsui Chemicals Corporation; trimethylol propane-adduct of xylylene diisocyanate containing NCO at 11.5 wt %), DESMODUR® L75 (Bayer; a polyisocyanate based on toluene diisocyanate), and DESMODUR® IL (Bayer; another polyisocyanate based on toluene diisocyanate).

The structures of certain commercially available polyisocyanates of the invention are shown below:

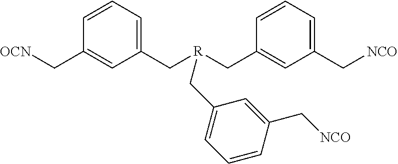

or its structural isomer. R can be a C₁-C₁₀ alkyl, C₁-C₁₀ ester, or an isocyanurate. Representative polyisocyanates having this structure are sold under the trademarks TAKENATE® D-110N (Mitsui), DESMODUR® L75 (Bayer), and DESMODUR® IL (Bayer).

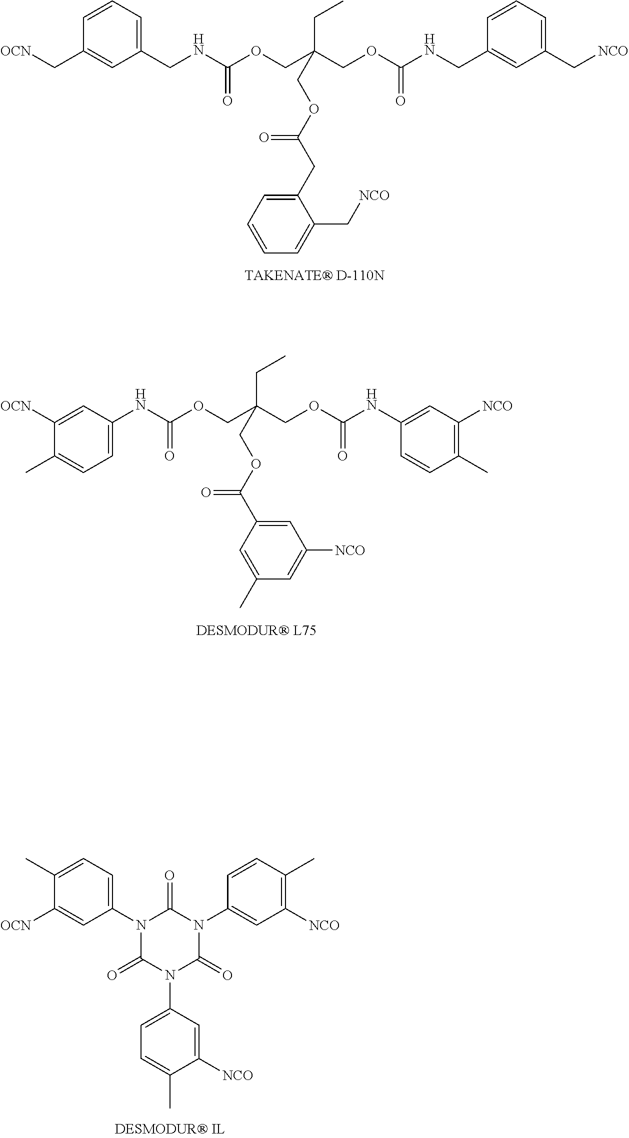

Polyisocyanate sold under the trademark TAKENATE® D-110N and other polyisocyanates are commercially available, typically in an ethyl acetate solution. Preferably, ethyl acetate is replaced with a solvent having a high flash point (e.g., at least 100° C., at least 120° C., and at least 150° C.). Suitable solvents include triacetin, triethyl citrate, ethylene glycol diacetate, and combinations thereof.

By way of illustration, a trimethylol propane-adduct of xylylene diisocyanate solution in ethyl acetate, which is sold under the trademark TAKENATE® D-110N, is combined with benzyl benzoate and vacuum distilled to remove ethyl acetate to obtain a polyisocyanate solution containing about 59% of the trimethylol propane-adduct of xylylene diisocyanate solution and 41% of benzyl benzoate. This polyisocyanate solution has a flash point of at least 60° C. This polyisocyanate solution in benzyl benzoate, together with polyvinylpyrrolidone/polyquaternium 11 or sulfonated polystyrene/carboxymethyl cellulose can be used to prepare the microcapsule composition of this invention. Such microcapsules have a performance similar to those made with the original trimethylol propane-adduct of xylylene diisocyanate solution in ethyl acetate sold under the trademark TAKENATE® D-110N.

Other specific examples of wall monomer isocyanates include 1,5-naphthylene diisocyanate, 4,4′-diphenylmethane diisocyanate (MDI), hydrogenated MDI (H12MDI), xylylene diisocyanate (XDI), tetramethylxylol diisocyanate (TMXDI), 4,4′-diphenyldimethylmethane diisocyanate, di- and tetraalkyldiphenylmethane diisocyanate, 4,4′-dibenzyl diisocyanate, 1,3-phenylene diisocyanate, 1,4-phenylene diisocyanate, the isomers of tolylene diisocyanate (TDI), 4,4′-diisocyanatophenylperfluoroethane, phthalic acid bisisocyanatoethyl ester, also polyisocyanates with reactive halogen atoms, such as 1-chloromethylphenyl 2,4-diisocyanate, 1-bromomethylphenyl 2,6-diisocyanate, and 3,3-bischloromethyl ether 4,4′-diphenyldiisocyanate, and combinations thereof.

In other embodiments, the polyisocyanate is an aliphatic polyisocyanate. In certain embodiments, the aliphatic polyisocyanate is a trimer of hexamethylene diisocyanate, a trimer of isophorone diisocyanate or a biuret of hexamethylene diisocyanate. Exemplary aliphatic polyisocyanates include those sold under the trademarks BAYHYDUR® N304 and BAYHYDUR® N305, which are aliphatic water-dispersible polyisocyanates based on hexamethylene diisocyanate; DESMODUR® N3600, DESMODUR® N3700, and DESMODUR® N3900, which are low viscosity, polyfunctional aliphatic polyisocyanates based on hexamethylene diisocyanate; and DESMODUR® 3600 and DESMODUR® N100 which are aliphatic polyisocyanates based on hexamethylene diisocyanate, each of which is available from Bayer Corporation (Pittsburgh, Pa.). More examples include 1-methyl-2,4-diisocyanatocyclohexane, 1,6-diisocyanato-2,2,4-trimethylhexane, 1,6-diisocyanato-2,4,4-trimethylhexane, 1-isocyanatomethyl-3-isocyanato-1,5,5-trimethylcyclohexane, chlorinated and brominated diisocyanates, phosphorus-containing diisocyanates, tetramethoxybutane 1,4-diisocyanate, butane 1,4-diisocyanate, hexane 1,6-diisocyanate (HDI), dicyclohexylmethane diisocyanate, cyclohexane 1,4-diisocyanate, ethylene diisocyanate, and combinations thereof. Sulfur-containing polyisocyanates are obtained, for example, by reacting hexamethylene diisocyanate with thiodiglycol or dihydroxydihexyl sulfide. Further suitable diisocyanates are trimethylhexamethylene diisocyanate, 1,4-diisocyanatobutane, 1,2-diisocyanatododecane, dimer fatty acid diisocyanate, and combinations thereof.

The average molecular weight of certain polyisocyanates useful in this invention varies from 250 Da to 1000 Da and preferable from 275 Da to 500 Da. In general, the range of the polyisocyanate concentration in the composition of this invention varies from 0.1% to 10%, preferably from 0.1% to 8%, more preferably from 0.2% to 5%, and even more preferably from 1.5% to 3.5%, all based on the total capsule composition.

In some embodiments, the polyfunctional isocyanate used in the preparation of the capsules of this invention is a single polyisocyanate. In other embodiments the polyisocyanate is a mixture of polyisocyanates. In some embodiments, the mixture of polyisocyanates includes an aliphatic polyisocyanate and an aromatic polyisocyanate. In particular embodiments, the mixture of polyisocyanates is a biuret of hexamethylene diisocyanate and a trimethylol propane-adduct of xylylene diisocyanate. In certain embodiments, the polyisocyanate is an aliphatic isocyanate or a mixture of aliphatic isocyanate, free of any aromatic isocyanate. In other words, in these embodiments, no aromatic isocyanate is used to prepare the polyurea/polyurethane polymers as capsule wall materials.

More examples of suitable isocyanates can be found in WO 2004/054362; EP 0148149; EP 0017409 B1; U.S. Pat. Nos. 4,417,916, 4,124,526, 5,583,090, 6,566,306, 6,730,635, WO 90/08468, WO 92/13450, U.S. Pat. Nos. 4,681,806, 4,285,720 and 6,340,653.

### Capsule Formation Aids

The microcapsule composition is typically prepared in the presence of a capsule formation aid, which can be a surfactant or dispersant. Capsule formation aids also improve the performance of the microcapsule composition. Performance is measured by the intensity of the fragrance released during certain stages, e.g., the pre-rub and post-rub phases in laundry applications. The pre-rub phase is the phase when the capsules have been deposited on the cloth, e.g., after a wash cycle using a capsule-containing fabric softener. The post-rub phase is after the capsules have been deposited and are broken by friction or other similar mechanisms.

In some embodiments, the capsule formation aid is a protective colloid or emulsifier including, e.g., maleic-vinyl copolymers such as the copolymers of vinyl ethers with maleic anhydride or acid, sodium lignosulfonates, maleic anhydride/styrene copolymers, ethylene/maleic anhydride copolymers, and copolymers of propylene oxide and ethylene oxide, polyvinylpyrrolidone (PVP), polyvinyl alcohols (PVA), sodium salt of naphthalene sulfonate condensate, carboxymethyl cellulose (CMC), fatty acid esters of polyoxyethylenated sorbitol, sodium dodecylsulfate, and combinations thereof. The surfactant concentration in the capsule composition varies from 0.1% to 5% (e.g., 0.5% to 4%, 0.2% to 2%, and 1% to 2%).

Commercially available surfactants include, but are not limited to, sulfonated naphthalene-formaldehyde condensates sold under the trademark MORWET® D425 (naphthalene sulfonate, Akzo Nobel, Fort Worth, Tex.); partially hydrolyzed polyvinyl alcohols sold under the trademark MOWIOL®, e.g., MOWIOL® 3-83 (Air Products); ethylene oxide-propylene oxide block copolymers or poloxamers sold under the trademarks PLURONIC®, SYNPERONIC® or PLURACARE® (BASF); sulfonated polystyrenes sold under the trademark FLEXAN® II (Akzo Nobel); ethylene-maleic anhydride polymers sold under the trademark ZEMAC® (Vertellus Specialties Inc.); and Polyquaternium series such as Polyquaternium 11 (“PQ11;” a copolymer of vinyl pyrrolidone and quaternized dimethylaminoethyl methacrylate; sold under the trademark LUVIQUAT® PQ11 AT 1 by BASF).

Processing aids can also be used as capsule formation aids. They include hydrocolloids, which improve the colloidal stability of the slurry against coagulation, sedimentation and creaming. The term “hydrocolloid” refers to a broad class of water-soluble or water-dispersible polymers having anionic, cationic, zwitterionic or non-ionic character. Hydrocolloids useful in the present invention include, but are not limited to, polycarbohydrates, such as starch, modified starch, dextrin, maltodextrin, and cellulose derivatives, and their quaternized forms; natural gums such as alginate esters, carrageenan, xanthans, agar-agar, pectins, pectic acid, and natural gums such as gum Arabic, gum tragacanth and gum karaya, guar gums and quaternized guar gums; gelatin, protein hydrolysates and their quaternized forms; synthetic polymers and copolymers, such as poly(vinyl pyrrolidone-co-vinyl acetate), poly(vinyl alcohol-co-vinyl acetate), poly((met)acrylic acid), poly(maleic acid), poly(alkyl(meth)acrylate-co-(meth)acrylic acid), poly(acrylic acid-co-maleic acid) copolymer, poly(alkyleneoxide), poly(vinyl-methylether), poly(vinylether-co-maleic anhydride), and the like, as well as poly-(ethyleneimine), poly((meth)acrylamide), poly(alkyleneoxide-co-dimethylsiloxane), poly(amino dimethylsiloxane), and the like, and their quaternized forms.

The capsule formation aid may also be used in combination with carboxymethyl cellulose (“CMC”), polyvinylpyrrolidone, polyvinyl alcohol, alkylnaphthalenesulfonate formaldehyde condensates, and/or a surfactant during processing to facilitate capsule formation. Examples of these surfactants include cetyl trimethyl ammonium chloride (CTAC); poloxamers sold under the trademarks PLURONIC® (e.g., PLURONIC® F127), PLURAFAC® (e.g., PLURAFAC® F127); a saponin sold under the trademark Q-NATURALE® (National Starch Food Innovation); or a gum Arabic such as Seyal or Senegal. In certain embodiments, the CMC polymer has a molecular weight range between about 90,000 Daltons to 1,500,000 Daltons, preferably between about 250,000 Daltons to 750,000 Daltons and more preferably between 400,000 Daltons to 750,000 Daltons. The CMC polymer has a degree of substitution between about 0.1 to about 3, preferably between about 0.65 to about 1.4, and more preferably between about 0.8 to about 1.0. The CMC polymer is present in the capsule slurry at a level from about 0.1% to about 2% and preferably from about 0.3% to about 0.7%. In other embodiments, polyvinylpyrrolidone used in this invention is a water-soluble polymer and has a molecular weight of 1,000 to 10,000,000. Suitable polyvinylpyrrolidone are polyvinylpyrrolidone K12, K15, K17, K25, K30, K60, K90, or a mixture thereof. The amount of polyvinylpyrrolidone is 2-50%, 5-30%, or 10-25% by weight of the capsule delivery system. A commercially available alkylnaphthalenesulfonate formaldehyde condensates is sold under the trademark MORWET® D-425, which is a sodium salt of naphthalene sulfonate condensate by Akzo Nobel (Fort Worth, Tex.).

### Catalysts

Catalysts suitable for use in the invention are metal carbonates, metal hydroxide, amino or organometallic compounds and include, for example, sodium carbonate, cesium carbonate, potassium carbonate, lithium hydroxide, 1,4-diazabicyclo[2.2.2]octane (i.e., DABCO), N,N-dimethylaminoethanol, N,N-dimethylcyclohexylamine, bis-(2-dimethylaminoethyl) ether, N,N dimethylacetylamine, stannous octoate and dibutyltin dilaurate.

### Other Encapsulating Polymers

In some other embodiments, the microcapsule of this invention has a microcapsule wall formed of a second encapsulating polymer selected from the group consisting of sol-gel polymer (e.g., silica), polyacrylate, polyacrylamide, poly(acrylate-co-acrylamide), polyurea, polyurethane, starch, gelatin and gum Arabic, poly(melamine-formaldehyde), poly(urea-formaldehyde), and combinations thereof. A branched polyethyleneimine is then coated onto the microcapsule wall to prepare a microcapsule having a positive zeta potential.

These encapsulating polymers are described in detail below.

Sol-Gel Microcapsules.

These microcapsules have a microcapsule wall formed of a sol-gel polymer, which is a reaction product of a sol-gel precursor via a polymerization reaction (e.g., hydrolyzation). Suitable sol-gel precursors are compounds capable of forming gels such as compounds containing silicon, boron, aluminum, titanium, zinc, zirconium, and vanadium. Preferred precursors are organosilicon, organoboron, and organoaluminum including metal alkoxides and b-diketonates.

Sol-gel precursors suitable for the purposes of the invention are selected in particular from the group of di-, tri- and/or tetrafunctional silicic acid, boric acid and alumoesters, more particularly alkoxysilanes (alkyl orthosilicates), and precursors thereof.

One example of sol-gel precursors suitable for the purposes of the invention are alkoxysilanes corresponding to the following general formula:

(R₁O)(R₂O)M(X)(X′),

wherein X can be hydrogen or —OR₃; X′ can be hydrogen or —OR₄; and R₁, R₂, R₃ and R₄ independently represent an organic group, more particularly a linear or branched alkyl group, preferably a C₁-C₁₂ alkyl. M can be Si, Ti, or Zr.

A preferred sol/gel precursor is alkoxysilanes corresponding to the following general formula: (R₁O)(R₂O)Si(X)(X′), wherein each of X, X′, R₁, and R₂ are defined above.

Particularly preferred compounds are the silicic acid esters such as tetramethyl orthosilicate (TMOS) and tetraethyl orthosilicate (TEOS). A preferred organofunctional silane is sold under the tradename Dynasylan® (Degussa Corporation, Parsippany, N.J.). Other sol-gel precursors suitable for the purposes of the invention are described, for example, in German Patent Application DE10021165. These sol-gel precursors are various hydrolyzable organosilanes such as, for example, alkylsilanes, alkoxysilanes, alkyl alkoxysilanes and organoalkoxysilanes. Besides the alkyl and alkoxy groups, other organic groups (for example allyl groups, aminoalkyl groups, hydroxyalkyl groups, etc.) may be attached as substituents to the silicon.

Recognizing that metal and semi-metal alkoxide monomers (and their partially hydrolyzed and condensed polymers) such as tetramethoxy silane (TMOS), tetraethoxy silane (TEOS), etc. are very good solvents for numerous molecules and active ingredients is highly advantageous since it facilitates dissolving the active materials at a high concentration and thus a high loading in the final capsules.

Polyacrylate Microcapsules, Polyacrylamide Microcapsules, and Poly(Acrylate-Co-Acrylamide) Microcapsules.

These microcapsules are prepared from corresponding precursors, which form the microcapsule wall. Preferred precursor are bi- or polyfunctional vinyl monomers including by way of illustration and not limitation, allyl methacrylate/acrylamide, triethylene glycol dimethacrylate/acrylamide, ethylene glycol dimethacrylate/acrylamide, diethylene glycol dimethacrylate/acrylamide, triethylene glycol dimethacrylate/acrylamide, tetraethylene glycol dimethacrylate/acrylamide, propylene glycol dimethacrylate/acrylamide, glycerol dimethacrylate/acrylamide, neopentyl glycol dimethacrylate/acrylamide, 1,10-decanediol dimethacrylate/acrylamide, pentaerythritol trimethacrylate/acrylamide, pentaerythritol tetramethacrylate/acrylamide, dipentaerythritol hexamethacrylate/acrylamide, triallyl-formal trimethacrylate/acrylamide, trimethylol propane trimethacrylate/acrylamide, tributanediol dimethacrylate/acrylamide, aliphatic or aromatic urethane diacrylates/acrylamides, difunctional urethane acrylates/acrylamides, ethoxylated aliphatic difunctional urethane methacrylates/acrylamides, aliphatic or aromatic urethane dimethacrylates/acrylamides, epoxy acrylates/acrylamides, epoxymethacrylates/acrylamides, 1,3-butylene glycol diacrylate/acrylamide, 1,4-butanediol dimethacrylate/acrylamide, 1,4-butaneidiol diacrylate/acrylamide, diethylene glycol diacrylate/acrylamide, 1,6-hexanediol diacrylate/acrylamide, 1,6-hexanediol dimethacrylate/acrylamide, neopentyl glycol diacrylate/acrylamide, polyethylene glycol diacrylate/acrylamide, tetraethylene glycol diacrylate/acrylamide, triethylene glycol diacrylate/acrylamide, 1,3-butylene glycol dimethacrylate/acrylamide, tripropylene glycol diacrylate/acrylamide, ethoxylated bisphenol diacrylate/acrylamide, ethoxylated bisphenol dimethylacrylate/acrylamide, dipropylene glycol diacrylate/acrylamide, alkoxylated hexanediol diacrylate/acrylamide, alkoxylated cyclohexane dimethanol diacrylate/acrylamide, propoxylated neopentyl glycol diacrylate/acrylamide, trimethylol-propane triacrylate/acrylamide, pentaerythritol triacrylate/acrylamide, ethoxylated trimethylolpropane triacrylate/acrylamide, propoxylated trimethylolpropane triacrylate/acrylamide, propoxylated glyceryl triacrylate/acrylamide, ditrimethyloipropane tetraacrylate/acrylamide, dipentaerythritol pentaacrylate/acrylamide, ethoxylated pentaerythritol tetraacrylate/acrylamide, PEG 200 dimethacrylate/acrylamide, PEG 400 dimethacrylate/acrylamide, PEG 600 dimethacrylate/acrylamide, 3-acryloyloxy glycol monoacrylate/acrylamide, triacryl formal, triallyl isocyanate, and triallyl isocyanurate.

The monomer is typically polymerized in the presence of an activation agent (e.g., an initiator) at a raised temperature (e.g., 30-90° C.) or under UV light. Exemplary initiators are 2,2′-azobis(isobutyronitrile) (“AIBN”), dicetyl peroxydicarbonate, di(4-tert-butylcyclohexyl) peroxydicarbonate, dioctanoyl peroxide, dibenzoyl peroxide, dilauroyl peroxide, didecanoyl peroxide, tert-butyl peracetate, tert-butyl perlaurate, tert-butyl perbenzoate, tert-butyl hydroperoxide, cumene hydroperoxide, cumene ethylperoxide, diisopropylhydroxy dicarboxylate, 2,2′-azobis(2,4-dimethylvaleronitrile), 1,1′-azobis-(cyclohexane-1-carbonitrile), dimethyl 2,2′-azobis(2-methylpropionate), 2,2′-azobis[2-methyl-N-(2-hydroxyethyl) propionamide, sodium persulfate, benzoyl peroxide, and combinations thereof.

Emulsifiers used in the formation of polyacrylate/polyacrylamide/poly(acrylate-co-acrylamide) capsule walls are typically anionic emulsifiers including by way of illustration and not limitation, water-soluble salts of alkyl sulfates, alkyl ether sulfates, alkyl isothionates, alkyl carboxylates, alkyl sulfosuccinates, alkyl succinamates, alkyl sulfate salts such as sodium dodecyl sulfate, alkyl sarcosinates, alkyl derivatives of protein hydrolyzates, acyl aspartates, alkyl or alkyl ether or alkylaryl ether phosphate esters, sodium dodecyl sulphate, phospholipids or lecithin, or soaps, sodium, potassium or ammonium stearate, oleate or palmitate, alkylarylsulfonic acid salts such as sodium dodecylbenzenesulfonate, sodium dialkylsulfosuccinates, dioctyl sulfosuccinate, sodium dilaurylsulfosuccinate, poly(styrene sulfonate) sodium salt, isobutylene-maleic anhydride copolymer, gum Arabic, sodium alginate, carboxymethylcellulose, cellulose sulfate and pectin, poly(styrene sulfonate), isobutylene-maleic anhydride copolymer, carrageenan, pectic acid, tragacanth gum, almond gum and agar; semi-synthetic polymers such as carboxymethyl cellulose, sulfated cellulose, sulfated methylcellulose, carboxymethyl starch, phosphated starch, lignin sulfonic acid; and synthetic polymers such as maleic anhydride copolymers (including hydrolysates thereof), polyacrylic acid, polymethacrylic acid, acrylic acid butyl acrylate copolymer or crotonic acid homopolymers and copolymers, vinylbenzenesulfonic acid or 2-acrylamido-2-methylpropanesulfonic acid homopolymers and copolymers, and partial amide or partial ester of such polymers and copolymers, carboxy-modified polyvinyl alcohol, sulfonic acid-modified polyvinyl alcohol and phosphoric acid-modified polyvinyl alcohol, phosphated or sulfated tristyrylphenol ethoxylates. The amount of anionic emulsifier is anywhere from 0.1 to 40 percent by weight of all constituents, more preferably from 0.5 to 10 percent, more preferably 0.5 to 5 percent by weight.

Aminoplasts and Gelatin.

A representative process used for aminoplast encapsulation is disclosed in U.S. Pat. No. 3,516,941 and US 2007/0078071, though it is recognized that many variations with regard to materials and process steps are possible. Another encapsulation process, i.e., gelatin encapsulation, is disclosed in U.S. Pat. No. 2,800,457. Both processes are discussed in the context of fragrance encapsulation for use in consumer products in U.S. Pat. Nos. 4,145,184 and 5,112,688, respectively. Polymer systems are well-known in the art and non-limiting examples of these include aminoplast capsules and encapsulated particles as disclosed in GB 2006709 A; the production of micro-capsules having walls comprising styrene-maleic anhydride reacted with melamine-formaldehyde precondensates as disclosed in U.S. Pat. No. 4,396,670; an acrylic acid-acrylamide copolymer, cross-linked with a melamine-formaldehyde resin as disclosed in U.S. Pat. No. 5,089,339; capsules composed of cationic melamine-formaldehyde condensates as disclosed in U.S. Pat. No. 5,401,577; melamine formaldehyde microencapsulation as disclosed in U.S. Pat. No. 3,074,845; amido-aldehyde resin in-situ polymerized capsules disclosed in EP 158 449 A1; etherified urea-formaldehyde polymer as disclosed in U.S. Pat. No. 5,204,185; melamine-formaldehyde microcapsules as described in U.S. Pat. No. 4,525,520; cross-linked oil-soluble melamine-formaldehyde precondensate as described in U.S. Pat. No. 5,011,634; capsule wall material formed from a complex of cationic and anionic melamine-formaldehyde precondensates that are then cross-linked as disclosed in U.S. Pat. No. 5,013,473; polymeric shells made from addition polymers such as condensation polymers, phenolic aldehydes, urea aldehydes or acrylic polymer as disclosed in U.S. Pat. No. 3,516,941; urea-formaldehyde capsules as disclosed in EP 0443428 A2; melamine-formaldehyde chemistry as disclosed in GB 2062570 A; and capsules composed of polymer or copolymer of styrene sulfonic acid in acid of salt form, and capsules cross-linked with melamine-formaldehyde as disclosed in U.S. Pat. No. 4,001,140.

Urea-Formaldehyde and Melamine-Formaldehyde Capsules.

Urea-formaldehyde and melamine-formaldehyde precondensate capsule shell wall precursors are prepared by means of reacting urea or melamine with formaldehyde where the mole ratio of melamine or urea to formaldehyde is in the range of from 10:1 to 1:6, preferably from 1:2 to 1:5. For purposes of practicing this invention, the resulting material has a molecular weight in the range of from 156 Da to 3000 Da. The resulting material may be used ‘as-is’ as a cross-linking agent for the aforementioned substituted or un-substituted acrylic acid polymer or copolymer or it may be further reacted with a C₁-C₆ alkanol, e.g., methanol, ethanol, 2-propanol, 3-propanol, 1-butanol, 1-pentanol or 1-hexanol, thereby forming a partial ether where the mole ratio of melamine/urea:formaldehyde:alkanol is in the range of 1:(0.1-6):(0.1-6). The resulting ether moiety-containing product may be used ‘as-is’ as a cross-linking agent for the aforementioned substituted or un-substituted acrylic acid polymer or copolymer, or it may be self-condensed to form dimers, trimers and/or tetramers which may also be used as cross-linking agents for the aforementioned substituted or un-substituted acrylic acid polymers or copolymers. Methods for formation of such melamine-formaldehyde and urea-formaldehyde pre-condensates are set forth in U.S. Pat. No. 6,261,483, and Lee et al. (2002) J. Microencapsulation 19:559-569.

Examples of urea-formaldehyde pre-condensates useful in the practice of this invention are sold under the trademarks URAC® 180 and URAC® 186. Examples of melamine-formaldehyde pre-condensates useful in the practice if this invention, include, but are not limited to, are melamine-formaldehyde pre-condensates sold under the trademarks CYMEL® U-60, CYMEL® U-64 and CYMEL® U-65 (Cytec Technology Corp.; Wilmington, Del.). It is preferable to use, as the precondensate for cross-linking, the substituted or unsubstituted acrylic acid polymer or co-polymer. In practicing this invention, the range of mole ratios of urea-formaldehyde precondensate/melamine-formaldehyde precondensate to substituted/un-substituted acrylic acid polymer/co-polymer is in the range of from 9:1 to 1:9, preferably from 5:1 to 1:5 and most preferably from 2:1 to 1:2.

In one embodiment of the invention, microcapsules with polymer(s) composed of primary and/or secondary amine reactive groups or mixtures thereof and cross-linkers can also be used. See US 2006/0248665. The amine polymers can possess primary and/or secondary amine functionalities and can be of either natural or synthetic origin. Amine-containing polymers of natural origin are typically proteins such as gelatin and albumen, as well as some polysaccharides. Synthetic amine polymers include various degrees of hydrolyzed polyvinyl formamides, polyvinylamines, polyallyl amines and other synthetic polymers with primary and secondary amine pendants. Examples of suitable polyvinylamines are sold under the trademark LUPAMIN® (BASF). The molecular weights of these materials can range from 10,000 Da to 1,000,000 Da.

Urea-formaldehyde or melamine-formaldehyde capsules can also include formaldehyde scavengers, which are capable of binding free formaldehyde. When the capsules are for use in aqueous media, formaldehyde scavengers such as sodium sulfite, melamine, glycine, and carbohydrazine are suitable. When the capsules are aimed to be used in products having low pH, e.g., fabric care conditioners, formaldehyde scavengers are preferably selected from beta diketones, such as beta-ketoesters, or from 1,3-diols, such as propylene glycol. Preferred beta-ketoesters include alkyl-malonates, alkyl aceto acetates and polyvinyl alcohol aceto acetates.

The microcapsule composition of this invention optionally contains one or more additional microcapsules, e.g., a second, third, fourth, fifth, or sixth microcapsules. Each of these microcapsules can be any of the microcapsule described above.

These additional microcapsules can be any of the microcapsules described above but different from each other in term of the microcapsule size, the degree of polymerization, the degree of crosslinking, the encapsulating polymer, the thickness of the wall, the active material, the ratio between the wall material and the active material, the rupture force or fracture strength, and the like.

### Active Materials

The core of the capsules of the invention can include one or more active materials including, but not limited to, flavors and/or fragrance ingredients such as fragrance oils. Individual active materials that can be encapsulated include those listed in WO 2016/049456, pages 38-50. These active material include flavor or fragrance ingredients, taste masking agents, taste sensates, malodor counteracting agents, vitamins or derivatives thereof, antibacterials, sunscreen actives, antioxidants, anti-inflammatory agents, fungicide, anesthetics, analgesics, antifungal agents, antibiotics, anti-viral agents, anti-parasitic agents, anti-infectious, anti-acne agents, dermatological active ingredients, enzymes and co-enzymes, skin whitening agents, anti-histamines, chemotherapeutic agents, insect repellents, emollient, skin moisturizing agent, wrinkle control agent, UV protection agent, fabric softener active, hard surface cleaning active, skin or hair conditioning agent, animal repellent, vermin repellent, flame retardant, antistatic agent, nanometer to micron size inorganic solid, polymeric or elastomeric particle, and combination thereof.

In addition to the active materials listed above, the products of this invention can also contain, for example, the following dyes, colorants or pigments: lactoflavin (riboflavin), beta-carotene, riboflavin-5′-phosphate, alpha-carotene, gamma-carotene, cantaxanthin, erythrosine, curcumin, quinoline yellow, yellow orange S, tartrazine, bixin, norbixin (annatto, orlean), capsanthin, capsorubin, lycopene, beta-apo-8′-carotenal, beta-apo-8′-carotenic acid ethyl ester, xanthophylls (flavoxanthin, lutein, cryptoxanthin, rubixanthin, violaxanthin, rodoxanthin), fast carmine (carminic acid, cochineal), azorubin, cochineal red A (Ponceau 4 R), beetroot red, betanin, anthocyanins, amaranth, patent blue V, indigotine I (indigo-carmine), chlorophylls, copper compounds of chlorophylls, acid brilliant green BS (lissamine green), brilliant black BN, vegetable carbon, titanium dioxide, iron oxides and hydroxides, calcium carbonate, aluminum, silver, gold, pigment rubine BK (lithol rubine BK), methyl violet B, victoria blue R, victoria blue B, acilan brilliant blue FFR (brilliant wool blue FFR), naphthol green B, acilan fast green 10 G (alkali fast green 10 G), ceres yellow GRN, sudan blue II, ultramarine, phthalocyanine blue, phthalocayanine green, fast acid violet R. Further naturally obtained extracts (for example paprika extract, black carrot extract, red cabbage extract) can be used for coloring purposes. Goods results are also achieved with the colors named in the following, the so-called aluminum lakes: FD & C Yellow 5 Lake, FD & C Blue 2 Lake, FD & C Blue 1 Lake, Tartrazine Lake, Quinoline Yellow Lake, FD & C Yellow 6 Lake, FD & C Red 40 Lake, victoria blue Lake, Carmoisine Lake, Amaranth Lake, Ponceau 4R Lake, Erythrosyne Lake, Red 2G Lake, Allura Red Lake, Patent Blue V Lake, Indigo Carmine Lake, Brilliant Blue Lake, Brown HT Lake, Black PN Lake, Green S Lake and mixtures thereof.

When the active material is a fragrance, it is preferred that fragrance ingredients within a fragrance having a C log P of 0.5 to 15 are employed. For instance, the ingredients having a C log P value between 0.5 to 8 (e.g., between 1 to 12, between 1.5 to 8, between 2 and 7, between 1 and 6, between 2 and 6, between 2 and 5, between 3 and 7) are 25% or greater (e.g., 50% or greater and 90% or greater) by the weight of the fragrance.

In some embodiments, it is preferred that a fragrance having a weight-averaged C log P of 2.5 and greater (e.g., 3 or greater, 2.5 to 7, and 2.5 to 5) is employed. The weight-averaged C log P is calculated as follows:

C log P={Sum[(Wi)(C log P)i]})/{Sum Wi},

in which Wi is the weight fraction of each fragrance ingredient and (C log P)i is the C log P of that fragrance ingredient.

As an illustration, it is preferred that greater than 60 weight percent, preferably greater than 80 and more preferably greater than 90 weight percent of the fragrance chemicals have C log P values of greater than 2, preferably greater than 3.3, more preferably greater than 4, and even more preferably greater than 4.5.

In other embodiments, the ingredients having a C log P value between 2 and 7 (e.g., between 2 and 6, and between 2 and 5) are 25% or greater (e.g., 50% or greater and 90% or greater) by the weight of the fragrance. It is preferred that greater than 60% (e.g., 80% or greater and 90% or greater) of the fragrance ingredients have Clog P values of greater than 3.3, preferably greater than 4 and most preferably greater than 4.5.

Those with skill in the art will appreciate that many fragrances can be created employing various solvents and fragrance chemicals. The use of a relatively low to intermediate C log P fragrance ingredients will result in fragrances that are suitable for encapsulation. These fragrances are generally water-insoluble, to be delivered through the capsule systems of this invention onto consumer products in different stages such as damp and dry fabric. Without encapsulation, the free fragrances would normally have evaporated or dissolved in water during use, e.g., wash. Though high log P materials are generally well delivered from a regular (non-encapsulated) fragrance in a consumer product, they have excellent encapsulation properties and are also suitable for encapsulation for overall fragrance character purposes, very long-lasting fragrance delivery, or overcoming incompatibility with the consumer product, e.g., fragrance materials that would otherwise be instable, cause thickening or discoloration of the product or otherwise negatively affect desired consumer product properties.

In some embodiments, the amount of encapsulated active material is from 5% to 95% (e.g., 20% to 90% and 40% to 85%) by weight of the capsule. The amount of the capsule wall is from 0.5% to 25% (e.g., 1.5% to 15% and 2.5% to 10%) also by weight of the capsule. In other embodiments, the amount of the encapsulated active material is from 15% to 99.5% (e.g., 50% to 98% and 30% to 95%) by weight of the capsule, and the amount of the capsule wall is from 0.5% to 85% (e.g., 2% to 50% and 5% to 70%) by weight of the capsule.

### Adjunct Materials

In addition to the active materials, the present invention also contemplates the incorporation of adjunct materials including solvent, emollients, and core modifier materials in the core encapsulated by the capsule wall. Other adjunct materials are solubility modifiers, density modifiers, stabilizers, viscosity modifiers, pH modifiers, or any combination thereof. These modifiers can be present in the wall or core of the capsules, or outside the capsules in delivery system. Preferably, they are in the core as a core modifier.

The one or more adjunct material may be added in the amount of from 0.01% to 25% (e.g., from 0.5% to 10%) by weight of the capsule.

Suitable examples include those described in WO 2016/049456, pages 55-57; and US 2016/0158121, pages 15-18.

### Deposition Aids

The copolymer of acrylamidopropyl trimonium chloride and acrylamide is used as the first deposition aid to facilitate the deposition of the microcapsule onto a hard surface (e.g., hair, skin, fiber, furniture, and floor). This copolymer for use in the invention generally has an average molecular weight (weight average molecular mass (MW) determined by size exclusion chromatography) of 2000 Da to 10000000 Da with a lower limit of 2000 Da, 5000 Da, 10000 Da, 20000 Da, 50000 Da, 100000 Da, 250000 Da, 500000 Da, or 800000 Da and an upper limit of 10000000 Da, 5000000 Da, 2000000 Da, 1000000 Da, or 500000 Da (e.g., 500000 Da to 2000000 Da and 800000 Da to 1500000 Da). The copolymer can also have a charge density ranging from 1 meq/g to 2.5 meq/g, preferably from 1.5 meq/g to 2.2 meq/g.

The copolymer of acrylamidopropyl trimonium chloride and acrylamide is commercially available from several vendors, e.g., sold under the trademark N-HANCE® SP-100 (Ashland). It is generally present at a level of 0.01% to 50% (with a lower limit of 0.01%, 0.05%, 0.1%, 0.5%, 1%, 2%, or 5% and an upper limit of 50%, 40%, 30%, 20%, 15%, or 10%, e.g., 0.1% to 30%, 1% to 20%, 2% to 15%, and 5% to 10%) by weight of the microcapsule composition. In a shampoo composition, the copolymer is generally present at a level of 0.001% to 20% (with a lower limit of 0.001%, 0.005%, 0.01%, 0.02%, or 0.05% and an upper limit of 20%, 15%, 10%, 5%, 2%, or 1%, e.g., 0.005% to 10%, 0.01% to 5%, and 0.02% to 0.5%) by weight of the shampoo composition.

A second capsule deposition aid from 0.01% to 25%, more preferably from 5% to 20% can be included by weight of the microcapsule composition. The capsule deposition aid can be added during the preparation of the capsules or it can be added after the capsules have been made.

These deposition aids are used to aid in deposition of capsules to surfaces such as fabric, hair or skin. These include anionically, cationically, nonionically, or amphoteric water-soluble polymers. Suitable deposition aids include polyquaternium-4, polyquaternium-5, polyquaternium-6, polyquaternium-7, polyquaternium-10, polyquaternium-16, polyquaternium-22, polyquaternium-24, polyquaternium-28, polyquaternium-39, polyquaternium-44, polyquaternium-46, polyquaternium-47, polyquaternium-53, polyquaternium-55, polyquaternium-67, polyquaternium-68, polyquaternium-69, polyquaternium-73, polyquaternium-74, polyquaternium-77, polyquaternium-78, polyquaternium-79, polyquaternium-80, polyquaternium-81, polyquaternium-82, polyquaternium-86, polyquaternium-88, polyquaternium-101, polyvinylamine, polyethyleneimine, polyvinylamine and vinylformamide copolymer, an acrylamidopropyl trimonium chloride/acrylamide (APTAC-Acm) copolymer, a methacrylamidopropyltrimethyl ammonium chloride/acrylamide (MAPTAC-Acm) copolymer, and combinations thereof. Other suitable deposition aids include those described in WO 2016/049456, pages 13-27. Additional deposition aids are described in US 2013/0330292, US 2013/0337023, and US 2014/0017278.

### Additional Components

The microcapsule composition of this invention can include one or more non-confined unencapsulated active materials from 0.01% to 50%, more preferably from 5% to 40%.

The capsule delivery system can also contain one or more other delivery system such as polymer-assisted delivery compositions (see U.S. Pat. No. 8,187,580), fiber-assisted delivery compositions (US 2010/0305021), cyclodextrin host guest complexes (U.S. Pat. No. 6,287,603 and US 2002/0019369), pro-fragrances (WO 2000/072816 and EP 0922084), and any combination thereof. The capsule delivery system can also contain one or more (e.g., two, three, four, five or six more) different capsules including different capsules of this invention and other capsules such as such as aminoplasts, hydrogel, sol-gel, coacervate capsules, polyurea/polyurethane capsules, and melamine formaldehyde capsules. More exemplary delivery systems that can be incorporated are coacervate capsules, cyclodextrin delivery systems, and pro-perfumes.

Examples of additional components include those described in US 2016/0158121, pages 24 to 25.

Any compound, polymer, or agent discussed above can be the compound, polymer, or agent itself as shown above, or its salt, precursor, hydrate, or solvate. A salt can be formed between an anion and a positively charged group on the compound, polymer, or agent. Suitable anions include chloride, bromide, iodide, sulfate, nitrate, phosphate, citrate, methanesulfonate, trifluoroacetate, acetate, malate, tosylate, tartrate, fumarate, glutamate, glucuronate, lactate, glutarate, and maleate. Likewise, a salt can also be formed between a cation and a negatively charged group on the compound, polymer, or agent. Suitable cations include sodium ion, potassium ion, magnesium ion, calcium ion, and an ammonium cation (e.g., tetramethylammonium ion). A precursor can be ester and another suitable derivative, which, during the process of preparing a polyurea or polyurethane capsule composition of this invention, is capable of converting to the compound, polymer, or agent and being used in preparing the polyurea or polyurethane capsule composition. A hydrate refers to the compound, polymer, or agent that contains water. A solvate refers to a complex formed between the compound, polymer, or agent and a suitable solvent. A suitable solvent can be water, ethanol, isopropanol, ethyl acetate, acetic acid, and ethanolamine.

Certain compounds, polymers, and agents have one or more stereocenters, each of which can be in the R configuration, the S configuration, or a mixture. Further, some compounds, polymers, and agents possess one or more double bonds wherein each double bond exists in the E (trans) or Z (cis) configuration, or combinations thereof. The compounds, polymers, and agents include all possible configurational stereoisomeric, regioisomeric, diastereomeric, enantiomeric, and epimeric forms as well as any mixtures thereof. As such, lysine used herein includes L-lysine, D-lysine, L-lysine monohydrochloride, D-lysine monohydrochloride, lysine carbonate, and so on. Similarly, arginine includes L-arginine, D-arginine, L-arginine monohydrochloride, D-arginine monohydrochloride, arginine carbonate, arginine monohydrate, and etc. Guanidine includes guanidine hydrochloride, guanidine carbonate, guanidine thiocyanate, and other guanidine salts including their hydrates. Ornithine includes L-ornithine and its salts/hydrates (e.g., monohydrochloride) and D-ornithine and its salts/hydrates (e.g., monohydrochloride).

The microcapsule composition of this invention can be a slurry containing in a solvent (e.g., water) the capsule at a level 0.1% to 80% (preferably 1% to 65% and more preferably 5% to 45%) by weight of the capsule delivery system.

In some embodiments, the microcapsule composition is purified by washing the capsule slurry with water until a neutral pH (pH of 6 to 8) is achieved. For the purposes of the present invention, the capsule suspension can be washed using any conventional method including the use of a separatory funnel, filter paper, centrifugation and the like. The capsule suspension can be washed one, two, three, four, five, six, or more times until a neutral pH, e.g., pH 6-8 and 6.5-7.5, is achieved. The pH of the purified capsules can be determined using any conventional method including, but not limited to pH paper, pH indicators, or a pH meter.

A capsule composition is “purified” in that it is 80%, 90%, 95%, 97%, 98% or 99% homogeneous to capsules. In accordance with the present invention, purity is achieved by washing the capsules until a neutral pH is achieved, which is indicative of removal of unwanted impurities and/or starting materials, e.g., polyisocyanate, cross-linking agent and the like.

In certain embodiments of this invention, the purification of the capsules includes the additional step of adding a salt to the capsule suspension prior to the step of washing the capsule suspension with water. Exemplary salts of use in this step of the invention include, but are not limited to, sodium chloride, potassium chloride or bi-sulphite salts. See US 2014/0017287.

The microcapsule composition of this invention can also be dried, e.g., spray dried, heat dried, and belt dried, to a solid form. In a spray drying process, a spray dry carrier is added to a microcapsule composition to assist the removal of water from the slurry. See US 2012/0151790, US 2014/0377446, US 2015/0267964, US 2015/0284189, and US 2016/0097591.

According to one embodiment, the spray dry carriers can be selected from the group consisting of carbohydrates such as chemically modified starches and/or hydrolyzed starches, gums such as gum arabic, proteins such as whey protein, cellulose derivatives, clays, synthetic water-soluble polymers and/or copolymers such as polyvinyl pyrrolidone, polyvinyl alcohol. The spray dry carriers may be present in an amount from 1% to 50%, more preferably from 5% to 20%.

Optionally, a free flow agent (anticaking agent) of silicas which may be hydrophobic silicas (i.e., silanol surface treated with halogen silanes, alkoxysilanes, silazanes, siloxanes, etc.) sold under the trademarks SIPERNAT® D17, AEROSIL® R972 and AEROSIL® R974 (available from Degussa) and/or hydrophilic silicas sold under the trademarks AEROSIL® 200, SIPERNAT® 22S, SIPERNAT® 50S, (available from Degussa), and SYLOID® 244 (available from Grace Davison), may be present from 0.01% to 10%, more preferable from 0.5% to 5%.

Humectants and viscosity control/suspending agents can also be added to facilitate spray drying. These agents are disclosed in U.S. Pat. Nos. 4,446,032 and 6,930,078. Details of hydrophobic silica as a functional delivery vehicle of active materials other than a free flow/anticaking agent are disclosed in U.S. Pat. Nos. 5,500,223 and 6,608,017.

The spray drying inlet temperature is in the range of 150° C. to 240° C., preferably between 170° C. and 230° C., more preferably between 190° C. and 220° C.

As described herein, the spray-dried microcapsule composition is well-suited for use in a variety of all dry (anhydrous) products: powder laundry detergent, fabric softener dryer sheets, household cleaning dry wipes, powder dish detergent, floor cleaning cloths, or any dry form of personal care products (e.g. shampoo powder, deodorant powder, foot powder, soap powder, baby powder), etc. Because of high fragrance and/or active agent concentration in the spray-dried products of the present invention, characteristics of the aforementioned consumer dry products will not be adversely affected by a small dosage of the spray-dried products.

The microcapsule composition can also be sprayed as a slurry onto a consumer product, e.g., a fabric care product. By way of illustration, a liquid delivery system containing capsules is sprayed onto a detergent powder during blending to make granules. See US 2011/0190191. In order to increase fragrance load, water-absorbing material, such as zeolite, can be added to the delivery system.

Alternatively, granulates in a consumer product are prepared in a mechanical granulator in the presence of a granulation auxiliary such as non-acid water-soluble organic crystalline solids. See WO 2005/097962.

### Zeta Potentials and Rupture Forces

The microcapsule of this invention is positively charged as indicated by a zeta potential of at least 10 mV, preferably at least 25 mV (e.g., 25 mV to 200 mV), and more preferably at least 40 mV (e.g., 40 mV to 100 mV).

Zeta potential is a measurement of electrokinetic potential in the microcapsule. From a theoretical viewpoint, zeta potential is the potential difference between the water phase (i.e., the dispersion medium) and the stationary layer of water attached to the surface of the microcapsule.

The zeta potential is an important indicator of the stability of the microcapsule in compositions or consumer products. Typically, a microcapsule having a zeta potential of 10 mV to 25 mV shows a moderate stability. Similarly, a microcapsule having a zeta potential of 25 mV to 40 mV shows a good stability and a microcapsule having a zeta potential of 40 mV to 100 mV shows excellent stability. Not to be bound by any theory, the microcapsule of this invention has a desirable zeta potential making it suitable for use in consumer products with improved stability.

Zeta potential is calculated using theoretical models and an experimentally-determined electrophoretic mobility or dynamic electrophoretic mobility. For more detailed discussion on measurement of zeta potential, see Dukhin & Goetz (2002) “Ultrasound for characterizing colloids,” Elsevier.

The microcapsule of this invention has a fracture strength of 0.2 MPa to 80 MPa (e.g., 0.5 MPa to 60 MPa, 1 MPa to 50 MPa, and 5 MPa to 30 MPa). The fracture strength of each microcapsule is calculated by dividing the rupture force (in Newtons) by the cross-sectional area of the respective microcapsule (πr2, where r is the radius of the particle before compression). The measurement of the rupture force and the cross-sectional area is performed following the methods described in Zhang et al. (2001) J. Microencapsul. 18(5):593-602.

The microcapsule of this invention has a rupture force of less than 10 mN (e.g., 0.1 mN to 10 mN, 0.2 mN to 8 mN, 0.3 mN to 5 mN, and 0.1 mN to 2 mN). The rupture force is the force needed to rupture the microcapsules. Its measurement is based on a technique known in the art as micro-manipulation. See Zhang et al. (1999) J. Microencapsul. 16(1):117-124.

### Hair Care Products

The microcapsule of this invention is especially suitable for use in hair care products including shampoo compositions and hair conditioning products.

Shampoo compositions typically contain detersive compositions, carriers, microcapsules, and deposition aids.

The detersive composition can be all aqueous phase or may comprise both an oil phase and an aqueous phase and may comprise any combination of the following components: a detersive surfactant, anti-dandruff agent, and aqueous carrier.

The detersive surfactant provides cleaning performance to the composition. The detersive surfactant in turn comprises anionic detersive surfactant, zwitterionic or amphoteric detersive surfactant, or combinations thereof. Various examples and descriptions of detersive surfactants are set forth in US 2016/0228338.

The concentration of the anionic surfactant component in the shampoo should be sufficient to provide the desired cleaning and lather performance, and generally ranges from 2% to 50% (e.g., 8% to 30%, 10% to 25%, and 12% to 22%.

Anionic surfactants suitable for use in the compositions are the alkyl and alkyl ether sulfates. Other suitable anionic detersive surfactants are the water-soluble salts of organic, sulfuric acid reaction products. Still other suitable anionic detersive surfactants are the reaction products of fatty acids esterified with isethionic acid and neutralized with sodium hydroxide. Examples include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In a further embodiment of the present invention, the anionic surfactant is sodium lauryl sulfate or sodium laureth sulfate.

Suitable amphoteric or zwitterionic detersive surfactants include those which are known for use in hair care or other personal care cleansing. Concentrations of such amphoteric detersive surfactants range from 0.5% to 20% (e.g., 1% to 10%). Non-limiting examples of suitable zwitterionic or amphoteric surfactants are described in U.S. Pat. Nos. 5,104,646 and 5,106,609. Examples of amphoteric detersive surfactants include cocoamphoacetate, cocoamphodiacetate, lauroamphoacetate, lauroamphodiacetate, and mixtures thereof. Zwitterionic detersive surfactants suitable for use in the composition include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. In another embodiment, zwitterionics such as betaines are selected. Non-limiting examples of other anionic, zwitterionic, amphoteric or optional additional surfactants suitable for use in the compositions are described in McCutcheon's, Emulsifiers and Detergents, 1989 Annual, published by M. C. Publishing Co., and U.S. Pat. No. 3,929,678.

The detersive composition may also contain an anti-dandruff agent. Examples include antimicrobial actives, pyridinethione salts, azoles, selenium sulfide, particulate sulfur, keratolytic acid, salicylic acid, octopirox (piroctone olamine), coal tar, and combinations thereof. In one aspect, the anti-dandruff agents typically are pyridinethione salts. Such anti-dandruff agents should be physically and chemically compatible with the essential components of the composition, and should not otherwise unduly impair product stability, aesthetics or performance.

The shampoo composition can be in the form of pourable liquids (under ambient conditions). Such compositions will therefore typically include an aqueous carrier, which is present at a level of from 20% to 95% (e.g., 60% to 85%). The aqueous carrier may comprise water, or a miscible mixture of water and organic solvent, and in one aspect may comprise water with minimal or no significant concentrations of organic solvent, except as otherwise incidentally incorporated into the composition as minor ingredients of other essential or optional components. The carrier useful in the present invention includes water and water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, hexylene glycol, glycerin, and propane diol.

The shampoo composition may further comprise other optional ingredients that are known for use or otherwise useful in compositions. Such optional ingredients are most typically those described in reference books such as the CTFA Cosmetic Ingredient Handbook, Second Edition, The Cosmetic, Toiletries, and Fragrance Association, Inc. 1988, 1992. Non-limiting examples of such optional ingredients include perfumes or fragrances, coloring agents or dyes, hair bleaching agents, thickeners, moisturizers, emollients, pharmaceutical actives, vitamins or nutrients, anti-dandruff agents, perfumes, hair colorants, hair perming agents, hair growth or restorer agents, and similar other materials.

The shampoo compositions can be in the form of rinse-off products or leave-on products, and can be formulated in a wide variety of product forms, including but not limited to creams, gels, emulsions, mousses and sprays.

In one embodiment, the shampoo composition is in the form of a gel comprising less than 45% water. In such embodiment, the gel may have a neat viscosity of 1000 cps to 10000 cps. The neat viscosity of a gel can be defined as the viscosity of the fluid at a shear rate of 1/sec. Scientifically, viscosity is the ratio of shear stress to shear rate. In some embodiments, the range of shear rates for gels is from 0.01/sec to 10/sec.

Hair conditioner products includes hair conditioners, leave-on hair conditioner, leave-in conditioner, rejuvenating conditioner, creme rinse, oil-free hair conditioners, rinse-off hair conditioner, conditioning rinse, foaming conditioner, conditioning styling gel, conditioning mousse, spay-on conditioner, hair dressing creme and hair repair spray.

The term “leave-on” refers to a hair care composition that is applied to the hair and not further subjected to a rinsing step. The term “rinse-out” as contrasted with the term “leave on” is used herein to mean compositions which are used in a context whereby the composition is ultimately rinsed or washed from the hair either after or during the application of the product.

Conditioning agents include any material used to give a particular conditioning benefit to hair. Suitable conditioning agents are those which deliver one or more benefits relating to shine, softness, comb ability, antistatic properties, wet-handling, damage, manageability, body, and greasiness. Examples include silicones (e.g., silicone oils, cationic silicones, silicone gums, high refractive silicones, silicone quaternary compounds, and silicone resins), organic conditioning oils (e.g., hydrocarbon oils, polyolefins, fatty acids, fatty alcohols, and fatty esters), alkyl quaternaries, and combinations thereof. See U.S. Pat. No. 6,696,053 and WO 2017/127494.

The concentration of the conditioning agent in the hair conditioner products should be sufficient to provide the desired conditioning benefits, and as will be apparent to one of ordinary skill in the art (e.g., 0.1% to 30%, 0.1% to 20%, 0.1% to 10%, and 0.1% to 6%).

The microcapsule composition can be present at a level of 0.02% to 15% (e.g., 0.05% to 10%, 0.1% to 5%, and 0.5% to 3%) so that the hair conditioning composition has a fragrance load of 0.01% to 5% (e.g., 0.02% to 3%, 0.05% to 2%, and 0.1% to 1%). The term “fragrance load” refers to the percentage of the fragrance by weight of the consumer product.

The hair care products can contain any one of the microcapsules described above and a conditioning agent. Optional additional components that can be included in the hair care products are cationic thickeners, carriers, emollients, moisturizing agents, hair soothing agents, anti-oxidants/radical scavengers, chelators or chelating agents, anti-inflammatory agents, antimicrobial actives, sunscreen actives, antidandruff agents, styling agents, hair bodying and volumizing agents, and combinations thereof. See U.S. Pat. No. 6,696,053 and WO 2017/127494.

### Fabric Conditioning Products

The microcapsule composition of this invention is also suitable for use in fabric care products such as fabric conditioning products.

The fabric conditioning compositions having the microcapsule composition contains at least one fabric conditioning agent, preferably at a concentration of 1% to 30% (e.g., 4% to 20%, 4% to 10%, and 8% to 15%). It would be obvious to a skilled person in the art to determine the concentration of a fabric conditioning agent while keeping its conditioning benefits and also maintaining a reasonable stability and shelf life.

Suitable fabric conditioning agents include cationic surfactants. Non-limiting examples are quaternary ammonium compounds such as alkylated quaternary ammonium compounds, ring or cyclic quaternary ammonium compounds, aromatic quaternary ammonium compounds, diquaternary ammonium compounds, alkoxylated quaternary ammonium compounds, amidoamine quaternary ammonium compounds, ester quaternary ammonium compounds, and mixtures thereof. Fabric softening compositions, and components thereof, are generally described in US 2004/0204337 and US 2003/0060390. Suitable softening agents include esterquats sold under the trademarks REWOQUAT® WE 18 by Evonik Industries and STEPANTEX® SP-90 by Stepan Company.

The microcapsule composition can be present at a level of 0.02% to 15% (e.g., 0.05% to 10%, 0.1% to 5%, and 0.5% to 3%) so that the fabric conditioning composition has a fragrance load of 0.01% to 5% (e.g., 0.02% to 3%, 0.05% to 2%, and 0.1% to 1%).

### Applications

The microcapsule of the present invention is well-suited for use, without limitation, in the following additional products presented in Table 1.

TABLE 1

| a) | Household products | |
|---|---|---|
| | i. | Liquid or Powder Laundry Detergents which can |

| | |
|---|---|
| | use the present invention include those systems |
| | described in U.S. Pat. No. 5,929,022, U.S. Pat. No. 5,916,862, |
| | U.S. Pat. No. 5,731,278, U.S. Pat. No. 5,565,145, U.S. Pat. No. |
| | 5,470,507, U.S. Pat. No. 5,466,802, U.S. Pat. No. 5,460,752, U.S. |
| | Pat. No. 5,458,810, U.S. Pat. No. 5,458,809, U.S. Pat. No. |
| | 5,288,431, U.S. Pat. No. 5,194,639, U.S. Pat. No. 4,968,451, |
| | U.S. Pat. No. 4,597,898, U.S. Pat. No. 4,561,998, U.S. Pat. No. |
| | 4,550,862, U.S. Pat. No. 4,537,707, U.S. Pat. No. 4,537,706, |
| | U.S. Pat. No. 4,515,705, U.S. Pat. No. 4,446,042, and U.S. Pat. |
| | No. 4,318,818. |
| ii. | Unit Dose Pouches, Tablets and Capsules such as |
| | those described in EP 1431382 A1, US 2013/0219996 |
| | A1, US 2013/0284637 A1, and U.S. Pat. No. 6,492,315. |
| | These unit dose formulations can contain high concentrations |
| | of a functional material (e.g., 5-100% fabric softening |
| | agent or detergent active), fragrance (e.g., 0.5%- |
| | 100%, 0.5%-40%, and 0.5%-15%), and flavor (e.g., |
| | 0.1%-100%, 0.1%-40%, and 1%-20%). They can contain |
| | no water to limit the water content as low as less |
| | than 30% (e.g., less than 20%, less than 10%, and |
| | less than 5%). |
| iii. | Scent Boosters such as those described in U.S. Pat. No. |
| | 7,867,968, U.S. Pat. No. 7,871,976, U.S. Pat. No. 8,333,289, |
| | US 2007/0269651 A1, and US2014/0107010 A1. |
| iv. | Fabric Care Products such as Rinse Conditioners |
| | (containing 1 to 30 weight % of a fabric |
| | conditioning active), Fabric Liquid Conditioners |
| | (containing 1 to 30 weight % of a fabric |
| | conditioning active), Tumble Drier Sheets, Fabric |
| | Refreshers, Fabric Refresher Sprays, Ironing |
| | Liquids, and Fabric Softener Systems such as those |
| | described in U.S. Pat. No. 6,335,315, U.S. Pat. No. 5,674,832, |
| | U.S. Pat. No. 5,759,990, U.S. Pat. No. 5,877,145, U.S. Pat. No. |
| | 5,574,179, U.S. Pat. No. 5,562,849, U.S. Pat. No. 5,545,350, |
| | U.S. Pat. No. 5,545,340, U.S. Pat. No. 5,411,671, U.S. Pat. No. |
| | 5,403,499, U.S. Pat. No. 5,288,417, U.S. Pat. No. 4,767,547 and |
| | U.S. Pat. No. 4,424,134. |
| | Liquid fabric softeners/fresheners contain at least |
| | one fabric softening agent present, preferably at a |
| | concentration of 1% to 30% (e.g., 4% to 20%, 4% to |
| | 10%, and 8% to 15%) . The ratio between the active |
| | material and the fabric softening agent can be 1:500 |
| | to 1:2 (e.g., 1:250 to 1:4 and 1:100 to 1:8). As an |
| | illustration, when the fabric softening agent is 5% |
| | by weight of the fabric softener, the active |
| | material is 0.01% to 2.5%, preferably 0.02% to 1.25% |
| | and more preferably 0.1% to 0.63%. As another |
| | example, when the fabric softening agent is 20% by |
| | weight of the fabric softener, the active material |
| | is 0.04% to 10%, preferably 0.08% to 5% and more |
| | preferably 0.4% to 2.5%. The active material is a |
| | fragrance, malodor counteractant or mixture thereof. |
| | The liquid fabric softener can have 0.15% to 15% of |
| | capsules (e.g., 0.5% to 10%, 0.7% to 5%, and 1% to |
| | 3%). When including capsules at these levels, the |
| | neat oil equivalent (NOE) in the softener is 0.05% |
| | to 5% (e.g., 0.15% to 3.2%, 0.25% to 2%, and 0.3% to |
| | 1%). |
| | Suitable fabric softening agents include cationic |
| | surfactants. Non-limiting examples are quaternary |
| | ammonium compounds such as alkylated quaternary |
| | ammonium compounds, ring or cyclic quaternary |
| | ammonium compounds, aromatic quaternary ammonium |
| | compounds, diquaternary ammonium compounds, |
| | alkoxylated quaternary ammonium compounds, |
| | amidoamine quaternary ammonium compounds, ester |
| | quaternary ammonium compounds, and mixtures thereof. |
| | Fabric softening compositions, and components |
| | thereof, are generally described in US 2004/0204337 |
| | and US 2003/0060390. Suitable softening agents |
| | include esterquats sold under the trademarks |
| | REWOQUAT ® WE 18 by Evonik Industries and |
| | STEPANTEX ® SP-90 by Stepan Company. |
| v. | Liquid dish detergents such as those described in |
| | U.S. Pat. No. 6,069,122 and U.S. Pat. No. 5,990,065 |
| vi. | Automatic Dish Detergents such as those described |
| | in U.S. Pat. No. 6,020,294, U.S. Pat. No. 6,017,871, U.S. Pat. |
| | No. 5,968,881, U.S. Pat. No. 5,962,386, U.S. Pat. No. 5,939,373, |
| | U.S. Pat. No. 5,914,307, U.S. Pat. No. 5,902,781, 5,705,464, |
| | U.S. Pat. No. 5,703,034, U.S. Pat. No. 5,703,030, U.S. Pat. No. |
| | 5,679,630, 5,597,936, U.S. Pat. No. 5,581,005, U.S. Pat. No. |
| | 5,559,261, U.S. Pat. No. 4,515,705, U.S. Pat. No. 5,169,552, |
| | and U.S. Pat. No. 4,714,562 |
| vii. | All-purpose Cleaners including bucket dilutable |
| | cleaners and toilet cleaners |
| viii. | Bathroom Cleaners |
| ix. | Bath Tissue |
| x. | Rug Deodorizers |
| xi. | Candles |
| xii. | Room Deodorizers |
| xiii. | Floor Cleaners |
| xiv. | Disinfectants |
| xv. | Window Cleaners |
| xvi. | Garbage bags/trash can liners |
| xvii. | Air Fresheners including room deodorizer and car |
| | deodorizer, scented candles, sprays, scented oil air |
| | freshener, Automatic spray air freshener, and |
| | neutralizing gel beads |
| xviii. | Moisture absorber |
| xix. | Household Devices such as paper towels and |
| | disposable Wipes |
| xx. | Moth balls/traps/cakes |

| b) | Baby Care Products | |
|---|---|---|
| | i. | Diaper Rash Cream/Balm |
| | ii. | Baby Powder |
| c) | Baby Care Devices | |
| | i. | Diapers |
| | ii. | Bibs |
| | iii. | Wipes |

| | |
|---|---|
| d) | Oral Care Products. Tooth care products (as an example |

of preparations according to the invention used for oral
care) generally include an abrasive system (abrasive or
polishing agent), for example silicic acids, calcium
carbonates, calcium phosphates, aluminum oxides and/or
hydroxylapatites, surface-active substances, for example
sodium lauryl sulfate, sodium lauryl sarcosinate and/or
cocamidopropylbetaine, humectants, for example glycerol
and/or sorbitol, thickening agents, for example
carboxymethyl cellulose, polyethylene glycols, carrageenan
and/or Laponite ®, sweeteners, for example saccharin, taste
correctors for unpleasant taste sensations, taste
correctors for further, normally not unpleasant taste
sensations, taste-modulating substances (for example
inositol phosphate, nucleotides such as guanosine
monophosphate, adenosine monophosphate or other substances
such as sodium glutamate or 2-phenoxypropionic acid),
cooling active ingredients, for example menthol
derivatives, (for example L-menthyllactate, L-
menthylalkylcarbonates, menthone ketals, menthane
carboxylic acid amides), 2,2,2-trialkylacetic acid amides
(for example 2,2-diisopropylpropionic acid methyl amide),
icilin and icilin derivatives, stabilizers and active
ingredients, for example sodium fluoride, sodium
monofluorophosphate, tin difluoride, quaternary ammonium
fluorides, zinc citrate, zinc sulfate, tin pyrophosphate,
tin dichloride, mixtures of various pyrophosphates,
triclosan, cetylpyridinium chloride, aluminum lactate,
potassium citrate, potassium nitrate, potassium chloride,
strontium chloride, hydrogen peroxide, flavorings and/or
sodium bicarbonate or taste correctors.

| | |
|---|---|
| i. | Tooth Paste. An exemplary formulation as |

| follows: | |
|---|---|
| 1. | calcium phosphate 40-55% |
| 2. | carboxymethyl cellulose 0.8-1.2% |
| 3. | sodium lauryl sulfate 1.5-2.5% |
| 4. | glycerol 20-30% |
| 5. | saccharin 0.1-0.3% |
| 6. | flavor oil 1-2.5% |
| 7. | water q.s. to 100% |

A typical procedure for preparing the formulation
includes the steps of (i) mixing by a blender
according to the foregoing formulation to provide a
toothpaste, and (ii) adding a composition of this
invention and blending the resultant mixture till
homogeneous.

| | ii. | Tooth Powder |
|---|---|---|
| | iii. | Oral Rinse |
| | iv. | Tooth Whiteners |
| | v. | Denture Adhesive |
| e) | Health Care Devices | |
| | i. | Dental Floss |
| | ii. | Toothbrushes |
| | iii. | Respirators |
| | iv. | Scented/flavored condoms |

| | |
|---|---|
| f) | Feminine Hygiene Products such as Tampons, Feminine |

Napkins and Wipes, and Pantiliners

| | |
|---|---|
| g) | Personal Care Products: Cosmetic or pharmaceutical |

preparations, e.g., a “water-in-oil” (W/O) type emulsion,
an “oil-in-water” (O/W) type emulsion or as multiple
emulsions, for example of the water-in-oil-in-water (W/O/W)
type, as a PIT emulsion, a Pickering emulsion, a micro-
emulsion or nano-emulsion; and emulsions which are
particularly preferred are of the “oil-in-water” (O/W) type
or water-in-oil-in-water (W/O/W) type. More specifically:

| | |
|---|---|
| i. | Personal Cleansers (bar soaps, body washes, and |

shower gels)

| | |
|---|---|
| ii. | In-shower conditioner |
| iii. | Sunscreen ant tattoo color protection (sprays, |

lotions, and sticks)

| | |
|---|---|
| iv. | Insect repellants |
| v. | Hand Sanitizer |
| vi. | Anti-inflammatory balms, ointments, and sprays |
| vii. | Antibacterial ointments and creams |
| viii. | Sensates |
| ix. | Deodorants and antiperspirants including |

aerosol and pump spray antiperspirant, stick
antiperspirant, roll-on antiperspirant, emulsion
spray antiperspirant, clear emulsion stick
antiperspirant, soft solid antiperspirant, emulsion
roll-on antiperspirant, clear emulsion stick
antiperspirant, opaque emulsion stick
antiperspirant, clear gel antiperspirant, clear
stick deodorant, gel deodorant, spray deodorant,
roll-on, and cream deodorant

| | |
|---|---|
| x. | Wax-based Deodorant. An exemplary formulation |

| as follows: | |
|---|---|
| 1. | Parafin Wax 10-20% |
| 2. | Hydrocarbon Wax 5-10% |
| 3. | White Petrolatum 10-15% |
| 4. | Acetylated Lanolin Alcohol 2-4% |
| 5. | Diisopropyl Adipate 4-8% |
| 6. | Mineral Oil 40-60% |
| 7. | Preservative (as needed) |

The formulation is prepared by (i) mixing the above
ingredients, (ii) heating the resultant composition
to 75° C. until melted, (iii) with stirring, adding
4% cryogenically ground polymer containing a
fragrance while maintaining the temperature 75° C.,
and (iv) stirring the resulting mixture in order to
ensure a uniform suspension while a composition of
this invention is added to the formulation.

| | |
|---|---|
| xi. | Glycol/Soap Type Deodorant. An exemplary |

| formulation as follows: | |
|---|---|
| 1. | Propylene Glycol 60-70% |
| 2. | Sodium Stearate 5-10% |
| 3. | Distilled Water 20-30% |
| 4. | 2,4,4-Trichloro-2′-Hydroxy Diphenyl Ether, |

0.01-0.5%
The ingredients are combined and heated to 75° C.
with stirring until the sodium stearate has
dissolved. The resulting mixture is cooled to 40° C.
followed by addition of a composition of this
invention.

| | |
|---|---|
| xii. | Lotion including body lotion, facial lotion, |

and hand lotion

| | xiii. | Body powder and foot powder |
|---|---|---|
| | xiv. | Toiletries |
| | xv. | Body Spray |
| | xvi. | Shave cream and male grooming products |
| | xvii. | Bath Soak |
| | xviii. | Exfoliating Scrub |
| h) | Personal Care Devices | |
| | i. | Facial Tissues |
| | ii. | Cleansing wipes |
| i) | Hair Care Products | |
| | i. | Shampoos (liquid and dry powder) |
| | ii. | Hair Conditioners (Rinse-out conditioners, |

leave-in conditioners, and cleansing conditioners)

| | |
|---|---|
| iii. | Hair Rinses |
| iv. | Hair Refreshers |
| v. | Hair perfumes |
| vi. | Hair straightening products |
| vii. | Hair styling products, Hair Fixative and |

styling aids

| | viii. | Hair combing creams |
|---|---|---|
| | ix. | Hair wax |
| | x. | Hair foam, hair gel, nonaerosol pump spray |
| | xi. | Hair Bleaches, Dyes and Colorants |
| | xii. | Perming agents |
| | xiii. | Hair wipes |
| j) | Beauty Care | |
| | i. | Fine Fragrance - Alcoholic. Compositions and |

methods for incorporating fragrance capsules into
alcoholic fine fragrances are described in U.S. Pat. No.
4,428,869. Alcoholic fine fragrances may contain the
following:

1. Ethanol (1-99%)
2. Water (0-99%)
3. A suspending aide including but not limited
to: hydroxypropyl cellulose, ethyl cellulose,
silica, microcrystalline cellulose, carrageenan,
propylene glycol alginate, methyl cellulose,
sodium carboxymethyl cellulose or xanthan gum
(0.1%)
4. Optionally an emulsifier or an emollient may
be included including but not limited to those
listed above.

| | |
|---|---|
| ii. | Solid Perfume |
| iii. | Lipstick/lip balm |
| iv. | Make-up cleanser |
| v. | Skin care cosmetic such as foundation, pack, |

sunscreen, skin lotion, milky lotion, skin cream,
emollients, skin whitening

| | |
|---|---|
| vi. | Make-up cosmetic including manicure, mascara, |

eyeliner, eye shadow, liquid foundation, powder
foundation, lipstick and cheek rouge

| | |
|---|---|
| k) | Consumer goods packaging such as fragranced cartons, |

fragranced plastic bottles/boxes

| l) | Pet care products | |
|---|---|---|
| | i. | Cat litter |
| | ii. | Flea and tick treatment products |
| | iii. | Pet grooming products |
| | iv. | Pet shampoos |
| | v. | Pet toys, treats, and chewables |
| | vi. | Pet training pads |
| | vii. | Pet carriers and crates |

| | |
|---|---|
| m) | Confectionaries confectionery, preferably selected from |

the group consisting of chocolate, chocolate bar products,
other products in bar form, fruit gums, hard and soft
caramels and chewing gum

| | |
|---|---|
| i. | Gum |

1. Gum base (natural latex chicle gum, most

current chewing gum bases also presently include
elastomers, such as polyvinylacetate (PVA),
polyethylene, (low or medium molecular weight)
polyisobutene (PIB), polybutadiene, isobutene-
isoprene copolymers (butyl rubber),
polyvinylethylether (PVE), polyvinylbutyether,
copolymers of vinyl esters and vinyl ethers,
styrene-butadiene copolymers (styrene-butadiene
rubber, SBR), or vinyl elastomers, for example based
on vinylacetate/vinyllaurate, vinylacetate/
vinylstearate or ethylene/vinylacetate, as well as
mixtures of the mentioned elastomers, as described
for example in EP 0 242 325, U.S. Pat. No.
4,518,615, U.S. Pat. No. 5, 093,136, U.S. Pat. No.
5,266,336, U.S. Pat. No. 5,601,858 or U.S. Pat. No.
6,986,709.) 20-25%

2. Powdered sugar 45-50%
3. glucose 15-17%
4. starch syrup 10-13%
5. plasticizer 0.1%
6. flavor 0.8-1.2%

The components described above were kneaded by a
kneader according to the foregoing formulation to
provide a chewing gum. Encapsulated Flavor or
sensate is then added and blended till homogeneous.

| | |
|---|---|
| ii. | Breath Fresheners |
| iii. | Orally Dissolvable Strips |
| iv. | Chewable Candy |
| v. | Hard Candy |

| | |
|---|---|
| n) | Baked products, preferably selected from the group |

consisting of bread, dry biscuits, cakes and other cookies

| | |
|---|---|
| o) | Snack foods, preferably selected from the group |

consisting of baked or fried potato chips or potato dough
products, bread dough products and corn or peanut-based
extrudates

| | |
|---|---|
| i. | Potato, tortilla, vegetable or multigrain chips |
| ii. | Popcorn |
| iii. | Pretzels |
| iv. | Extruded stacks |

| | |
|---|---|
| p) | Cereal Products preferably selected from the group |

consisting of breakfast cereals, muesli bars and precooked
finished rice products

| | |
|---|---|
| q) | Alcoholic and non-alcoholic beverages, preferably |

selected from the group consisting of coffee, tea, wine,
beverages containing wine, beer, beverages containing beer,
liqueurs, schnapps, brandies, sodas containing fruit,
isotonic beverages, soft drinks, nectars, fruit and
vegetable juices and fruit or vegetable preparations;
instant beverages, preferably selected from the group
consisting of instant cocoa beverages, instant tea
beverages and instant coffee beverages

| | |
|---|---|
| i. | Ready to drink liquid drinks |
| ii. | Liquid Drink Concentrates |
| iii. | Powder Drinks |
| iv. | Coffee: Instant Cappuccino |

| | |
|---|---|
| 1. | Sugar 30-40% |
| 2. | Milk Powder 24-35% |
| 3. | Soluble Coffee 20-25% |
| 4. | Lactose 1-15% |
| 5. | Food Grade Emulsifier 1-3% |
| 6. | Encapsulated Volatile Flavor 0.01-0.5% |

| | v. | Tea |
|---|---|---|
| | vi. | Alcoholics |
| r) | Spice blends and consumer prepared foods | |
| | i. | Powder gravy, sauce mixes |
| | ii. | Condiments |
| | iii. | Fermented Products |

| | |
|---|---|
| s) | Ready to heat foods: ready meals and soups, preferably |

selected from the group consisting of powdered soups,
instant soups, precooked soups

| | |
|---|---|
| i. | Soups |
| ii. | Sauces |
| iii. | Stews |
| iv. | Frozen entrees |

| | |
|---|---|
| t) | Dairy Products milk products, preferably selected from |

the group consisting of milk beverages, ice milk, yogurt,
kefir, cream cheese, soft cheese, hard cheese, powdered
milk, whey, butter, buttermilk and partially or fully
hydrolyzed milk protein-containing products Flavored milk
beverages

| | |
|---|---|
| i. | Yoghurt |
| ii. | Ice cream |
| iii. | Bean Curd |
| iv. | Cheese |

| | |
|---|---|
| u) | Soya protein or other soybean fractions, preferably |

selected from the group consisting of soya milk and
products produced therefrom, soya lecithin-containing
preparations, fermented products such as tofu or tempeh or
products produced therefrom and soy sauces

| | |
|---|---|
| v) | Meat products, preferably selected from the group |

consisting of ham, fresh or raw sausage preparations, and
seasoned or marinated fresh or salt meat products

| | |
|---|---|
| w) | Eggs or egg products, preferably selected from the group |

consisting of dried egg, egg white and egg yolk

| | |
|---|---|
| x) | Oil-based products or emulsions thereof, preferably |

selected from the group consisting of mayonnaise,
remoulade, dressings and seasoning preparations

| | |
|---|---|
| y) | fruit preparations, preferably selected from the group |

consisting of jams, sorbets, fruit sauces and fruit
fillings; vegetable preparations, preferably selected from
the group consisting of ketchup, sauces, dried vegetables,
deep-frozen vegetables, precooked vegetables, vegetables in
vinegar and preserved vegetables

| | |
|---|---|
| z) | Flavored pet foods |

The above-listed applications are all well-known in the art. For example, fabric softener systems are described in U.S. Pat. Nos. 6,335,315, 5,674,832, 5,759,990, 5,877,145, 5,574,179, 5,562,849, 5,545,350, 5,545,340, 5,411,671, 5,403,499, 5,288,417, 4,767,547, and 4,424,134. Liquid laundry detergents include those systems described in U.S. Pat. Nos. 5,929,022, 5,916,862, 5,731,278, 5,565,145, 5,470,507, 5,466,802, 5,460,752, 5,458,810, 5,458,809, 5,288,431, 5,194,639, 4,968,451, 4,597,898, 4,561,998, 4,550,862, 4,537,707, 4,537,706, 4,515,705, 4,446,042, and 4,318,818. Liquid dish detergents are described in U.S. Pat. Nos. 6,069,122 and 5,990,065. Shampoo and conditioners that can employ the present invention include those described in U.S. Pat. Nos. 6,162,423, 5,968,286, 5,935,561, 5,932,203, 5,837,661, 5,776,443, 5,756,436, 5,661,118, 5,618,523, 5,275,755, 5,085,857, 4,673,568, 4,387,090 and 4,705,681. Automatic Dish Detergents are described in U.S. Pat. Nos. 6,020,294, 6,017,871, 5,968,881, 5,962,386, 5,939,373, 5,914,307, 5,902,781, 5,705,464, 5,703,034, 5,703,030, 5,679,630, 5,597,936, 5,581,005, 5,559,261, 4,515,705, 5,169,552, and 4,714,562.

All parts, percentages and proportions refer to herein and in the claims are by weight unless otherwise indicated.

The values and dimensions disclosed herein are not to be understood as being strictly limited to the exact numerical values recited. Instead, unless otherwise specified, each such value is intended to mean both the recited value and a functionally equivalent range surrounding that value. For example, a value disclosed as “50%” is intended to mean “about 50%.”

The terms “include,” “includes,” and “including,” are meant to be non-limiting.

The terms “capsule” and “microcapsule” herein are used interchangeably.

The terms “polyfunctional isocyanate,” “multifunctional isocyanate,” and “polyisocyanate” are used interchangeably and refer to a compound having two or more isocyanate (—NCO) groups.

The terms “polyfunctional amine,” “multifunctional amine,” and “polyamine” are used interchangeably and refer to a compound containing one, two, or more primary or secondary amine groups. These terms also refer to a compound containing one or more primary/secondary amine groups and one or more hydroxyl groups (—OH).

The terms “polyethyleneimine,” “polyethyleneimines,” “polyethylenimine,” and “polyethylenimines” are used interchangeably.

The terms “polyfunctional alcohol,” “multifunctional alcohol,” “poly alcohol,” and “polyol” are used interchangeably and refer to a compound having two or more hydroxyl groups.

The term “degree of polymerization” refers to the number of repeat units in a polymer.

The term “degree of crosslinking” refers to percent of interconnecting units over the total repeat unit. It is generally measured by swelling experiments. See ASTM Standard Test Method ASTM D2765-11; Lange (1986) Colloid & Polymer Science 264:488-93.

The terms “multi-functional nucleophile” and “polyfunctional nucleophile” are used herein interchangeably. They both refer to an aliphatic or aromatic hydrocarbon onto which is attached two or more nucleophilic groups such as primary/secondary amine groups and the hydroxyl group.

The term “multi-functional electrophile” and “polyfunctional electrophile” are used interchangeably and refer to an aliphatic or aromatic hydrocarbon, onto which is attached two or more electrophilic groups reactive towards the nucleophilic group. Examples of an electrophilic group include: aldehydes, halide, sulfate esters, sulphonate esters, epoxide, chlorohydrins as well as terminal olefins conjugated with a carbonyl group including ketone, amide, or ester.

The terms “acrylamidopropyltrimonium chloride/acrylamide copolymer” and “copolymer of acrylamide and acrylamidopropyltrimonium chloride” are used interchangeably.

The invention is described in greater detail by the following non-limiting examples. Without further elaboration, it is believed that one skilled in the art can, based on the description herein, utilize the present invention to its fullest extent. All publications cited herein are incorporated by reference in their entirety.

### Example 1: Improved Deposition of Capsules in Shampoo Base

A microcapsule slurry of this invention, i.e., Microcapsule Slurry A, was prepared following the procedure described below. Microcapsule Slurry A was used to prepare microcapsule compositions and shampoo compositions of this invention.

Microcapsule Slurry A.

An oil phase was provided by mixing 96 grams (“g”) of a research fragrance accord, 24 g of caprylic/capric triglyceride oil sold under the trademark NEOBEE® M-5 (Stepan, Chicago, Ill.), and 9.6 g of trimethylol propane-adduct of xylylene diisocyanate sold under the trademark TAKENATE® D110-N (Mitsui Chemicals Corporation, Rye Brook, N.Y.). In a separate beaker, an aqueous phase was prepared by mixing an aqueous solution (130 g) containing 1.2% of polystyrene sulfonate sold under the trademark FLEXAN® II (Akzo Nobel, Bridgewater, N.J.) and an aqueous solution (30 g) of 1% CMC (carboxymethyl cellulose sold under the trademark WALOCEL® CRT 50000 PA 07; Dow, Midland, Mich.). The oil phase was emulsified into the aqueous phase to form an oil-in-water fragrance emulsion under shearing (ULTRA TURRAX, T25 Basic, IKA WERKE) at 9500 rpm for two minutes. After the fragrance emulsion was heated to 25° C., 10.4 g of a 49% branched polyethylenimine (Sigma-Aldrich, St. Louis, Mo.) aqueous solution was added under constant mixing with an overhead mixer. Formation of capsules was immediately visible by optical microscopy. The resultant capsule slurry was cured at 55° C. for two hours to obtain Microcapsule Slurry A. Microcapsule Slurry A contained a polyurea microcapsule having a microcapsule core and a polyurea microcapsule wall.

Microcapsule Compositions 1-5.

Five microcapsule compositions, i.e., Microcapsule Compositions 1-5, were prepared by mixing Microcapsule Slurry A, water, and an acrylamidopropyltrimonium chloride/acrylamide copolymer (sold under the trademark N-HANCE® SP-100; Ashland). The amount of each component (by weight) is shown in Table 2. Each composition contained about 9% of the polyurea microcapsule, or 19.5% of Microcapsule Slurry A, both by weight of the microcapsule composition. The acrylamidopropyltrimonium chloride/acrylamide copolymer (APTAC-Acm) was used as a 10 wt % aqueous solution.

TABLE 2

| | | APTAC-Acm solution, | |
|---|---|---|---|
| Microcapsule | Microcapsule | g (% by weight of | Water, |
| Composition | Slurry A, g | the composition) | g |
| 1 | 0.78 | 0 | 3.22 |
| 2 | 0.78 | 0.5 (1.25) | 2.72 |
| 3 | 0.78 | 1 (2.5) | 2.22 |
| 4 | 0.78 | 1.5 (3.75) | 1.72 |
| 5 | 0.78 | 2.5 (6.25) | 0.72 |

Shampoo Compositions 1-5.

Five shampoo compositions of this invention, i.e., Shampoo Compositions 1-5, were prepared by mixing each of Microcapsule Compositions 1-5 (4 g) with 96 g of a shampoo base (unperfumed SANEX® Men Zero % shampoo, commercially available from Colgate Palmolive, N.Y.). Each shampoo was macerated for at least 24 hours at room temperature before evaluation.

Shampoo Performance.

Each shampoo composition thus prepared was applied to five wet hair swatches with excess water squeezed out. The hair swatches were then lathered, rinsed with water, and, optionally activated with a brush. A trained panel of judges evaluated the fragrance intensity on a scale ranging from 0 to 10. A numerical value of 5 indicated the hair swatches produced a strong intensity, while a value of 10 indicated the hair swatches generated a very strong smell. One hair swatch was evaluated without activation and the other was evaluated with activation.

Deposition Evaluation.

A treated wet hair swatch was extracted with methanol to analyze deposition efficiency using a gas chromatography-mass spectrometry (GC-MS) instrument. The results of deposition and fragrance intensity are provided in Table 3.

TABLE 3

| | Deposition relative | Intensity | Intensity |
|---|---|---|---|
| Shampoo | to Shampoo | before | after |
| Composition | Composition 1 | activation | activation |
| 1 | 1 | 0.5 | 4 |
| 2 | 1.8 | 1.5 | 6.5 |
| 3 | 2.7 | 1 | 7 |
| 4 | 6.8 | 1 | 8.5 |
| 5 | 4.6 | 1 | 7 |

As shown in Table 3 above, Microcapsule Compositions 2-5 each had (i) a deposition 1.8- to 6.8-fold that of the microcapsules in Shampoo Composition 1 and (ii) a fragrance intensity much greater than Shampoo Composition 1.

### Example 2: Methods for Preparing Shampoo Compositions

Acrylamidopropyltrimonium chloride/acrylamide (APTAC-Acm) copolymer can be incorporated at different stages of the shampoo composition preparation. The copolymer can be added either (a) as a coating of the microcapsules in the microcapsule slurry, namely as a component of the microcapsule composition, (b) by adding the copolymer to the shampoo base (i.e., the detersive composition) together with microcapsules, (c) in the process of the shampoo manufacturing without the microcapsules, or (d) in the process of the shampoo manufacturing with the microcapsules.

Four additional shampoo compositions were prepared (Shampoo Compositions 6-9), which incorporated Microcapsule Slurry A. The general steps for obtaining Shampoo Compositions 6-9, which incorporated Microcapsule Slurry A, are as follows. The specific steps for obtaining Shampoo Compositions 6-9 are presented in Tables 4-7.

General Procedure for Preparing Shampoo Compositions:

(a) In a beaker, a first mixture was obtained by mixing a behenyl alcohol sold under the trademark LANETTE® 22, a distearyl ether sold under the trademark CUTINA® STE, and Cocamide MIPA sold under the trademark REWOMID® IPP 240 at 75° C.;

(b) In another beaker, an acrylate copolymer sold under the trademark CARBOPOL® was mixed in water. Surfactant sold under the trademark TEXAPON® was then added at 75° C. The pH of the resultant mixture was adjusted to neutral (pH 6.5-7.5) with a NaOH solution to obtain a second mixture;

(c) A third mixture was obtained by mixing the first mixture and the second mixture;

(d) A fourth mixture was prepared by mixing the third mixture at 40° C. with concentrated cocamidopropyl betaine sold under the trademark TEGO® Betaine F50, an organosilicon sold under the trademark BELSIL®, Kathon™ CG preservative (isothiazolinone), and Citric Acid solution in any order. In some examples, this fourth mixture was the final shampoo composition when the microcapsule and APTAC-Acm copolymer were added in any of the steps (a-d); and

(e) Optionally, the shampoo composition was obtained by adding the microcapsule composition and the APTAC-Acm copolymer. Water was added when mentioned in Tables 4-7.

Preparation of Shampoo Composition 6.

To prepare Shampoo Composition 6, Microcapsule Composition 6 was added in Step (e) as provided in Table 4. Microcapsule Composition 6 was obtained by mixing 100 g of Microcapsule Slurry A (Example 1) and 42.86 g of the APTAC-Acm copolymer (sold under the trademark N-HANCE® SP-100; Ashland).

TABLE 4

| Step | Component | wt % |
|---|---|---|
| (a) | Cocamide MIPA sold under the trademark | 1.0 |
| | REWOMID ® IPP 240 (cocamide | |
| | monoisopropanolamine, CAS # 68333-82-4; Evonik | |
| | Industries AG Personal Care, Germany) | |
| | Behenyl alcohol sold under the trademark | 1.0 |
| | LANETTE ® 22 (BASF, Florham Park, NJ) | |
| | Distearyl ether sold under the trademark | 1.0 |
| | CUTINA ® STE (BASF, Florham Park, NJ) | |
| (b) | Acrylate copolymer sold under the trademark | 5.0 |
| | CARBOPOL ® Aqua SF-1 (Lubrizol, Wickliffe, | |
| | Ohio) | |
| | Water | 55.0 |
| | Sodium laureth sulfate sold under the | 12.9 |
| | trademark TEXAPON ® N70 (BASF, Florham Park, | |
| | NJ) | |
| | Sodium hydroxide (NaOH, 18% aqueous solution; | 0.9 |
| | VWR International, Radnor, PA) | |
| (c) | Mixing (a) and (b) Sub-total | 76.8 |
| (d) | Cocamidopropyl betaine sold under the | 7.9 |
| | trademark TEGO ® Betaine F50 (Evonik, | |
| | Parsippany-Troy Hills, NJ) | |
| | Dimethiconol & TEA-Dodecylbenzenesulfonate | 2.0 |
| | sold under the trademark BELSIL ® DM 60 10 | |
| | (Wacker Chemie AG, Calvert City, KY) | |
| | Methylchloroisothiazolinone and | 0.1 |
| | Methylisothiazolinone (Kathon ™ CG; Brenntag | |
| | North America, Reading, PA) | |
| | Citric acid (sol at 10% in H₂O; VWR) | 3.1 |
| | Water | 5.1 |
| (e) | Water | 3.88 |
| | Microcapsule Composition 6 | 1.12 |
| | Total | 100 |

Preparation of Shampoo Composition 7.

Shampoo Composition 7 was prepared following the general procedure described above except that Microcapsule Slurry A was added in Step (e) into the third mixture, followed by the addition of the APTAC-Acm copolymer as outlined in Table 5.

TABLE 5

| Step | Component | wt % |
|---|---|---|
| (a) | Cocamide MIPA sold under the trademark | 1.0 |
| | REWOMID ® IPP 240 (Evonik) | |
| | Behenyl alcohol sold under the trademark | 1.0 |
| | LANETTE ® 22 (BASF, Florham Park, NJ) | |
| | Distearyl ether sold under the trademark | 1.0 |
| | CUTINA ® STE (BASF) | |
| (b) | Acrylate copolymer sold under the trademark | 5.0 |
| | CARBOPOL ® Aqua SF-1 (Lubrizol) | |
| | Water | 55.0 |
| | Sodium laureth sulfate sold under the | 12.9 |
| | trademark TEXAPON ® N70 (BASF) | |
| | NaOH (18% aqueous solution; VWR) | 0.9 |
| (c) | Mixing (a) and (b) Sub-total | 76.8 |
| (d) | Cocamidopropyl betaine sold under the | 7.9 |
| | trademark TEGO ® Betaine F50 (Evonik) | |
| | Dimethiconol & TEA-Dodecylbenzenesulfonate | 2.0 |
| | sold under the trademark BELSIL ® DM 60 10 | |
| | (Wacker) | |
| | Methylchloroisothiazolinone and | 0.1 |
| | Methylisothiazolinone (Kathon ™ CG; Brenntag) | |
| | Citric acid (10% aqueous solution; VWR) | 3.1 |
| | Water | 5.1 |
| (e) | Water | 1.72 |
| | Microcapsule Slurry A | 0.78 |
| | APTAC-Acm copolymer sold under the trademark | 2.5 |
| | N-HANCE ® SP-100 (10% aqueous solution; | |
| | Ashland) | |
| | Total | 100 |

Preparation of Shampoo Composition 8.

Shampoo Composition 8 was prepared following the general procedure described above except that the APTAC-Acm copolymer was added in Step (d) into the third mixture and Microcapsule Slurry A was added after Step (e) into the fourth mixture as outlined in Table 6.

TABLE 6

| Step | Component | wt % |
|---|---|---|
| (a) | Cocamide MIPA sold under the trademark | 1.0 |
| | REWOMID ® IPP 240 (Evonik) | |
| | Behenyl alcohol sold under the trademark | 1.0 |
| | LANETTE ® 22 (BASF, Florham Park, NJ) | |
| | Distearyl ether sold under the trademark | 1.0 |
| | CUTINA ® STE (BASF) | |
| (b) | Acrylate copolymer sold under the trademark | 5.0 |
| | CARBOPOL ® Aqua SF-1 (Lubrizol) | |
| | Water | 55.0 |
| | Sodium laureth sulfate sold under the | 12.9 |
| | trademark TEXAPON ® N70 (BASF) | |
| | NaOH (18% aqueous solution; VWR) | 0.9 |
| (c) | Mixing (a) and (b) Sub-total | 76.8 |
| (d) | APTAC-Acm copolymer sold under the trademark | 2.5 |
| | N-HANCE ® SP-100 (10% aqueous solution; | |
| | Ashland) | |
| | Cocamidopropyl betaine sold under the | 7.9 |
| | trademark TEGO ® Betaine F50 (Evonik) | |
| | Dimethiconol & TEA-Dodecylbenzenesulfonate | 2.0 |
| | sold under the trademark BELSIL ® DM 60 10 | |
| | (Wacker) | |
| | Methylchloroisothiazolinone and | 0.1 |
| | Methylisothiazolinone (Kathon ™ CG; Brenntag) | |
| | Citric acid (10% aqueous solution; VWR) | 3.1 |
| | Water | 2.6 |
| (e) | Water | 4.22 |
| | Microcapsule Slurry A | 0.78 |
| | Total | 100 |

Preparation of Shampoo Composition 9.

Shampoo Composition 9 was prepared following the general procedure described above except that the APTAC-Acm copolymer and Microcapsule Slurry A were added in Step (d) into the third mixture as outlined in Table 7.

TABLE 7

| Step | Component | wt % |
|---|---|---|
| (a) | Cocamide MIPA sold under the trademark | 1.0 |
| | REWOMID ® IPP 240 (Evonik) | |
| | Behenyl alcohol sold under the trademark | 1.0 |
| | LANETTE ® 22 (BASF, Florham Park, NJ) | |
| | Distearyl ether sold under the trademark | 1.0 |
| | CUTINA ® STE (BASF) | |
| (b) | Acrylate copolymer sold under the trademark | 5.0 |
| | CARBOPOL ® Aqua SF-1 (Lubrizol) | |
| | Water | 55.0 |
| | Sodium laureth sulfate sold under the | 12.9 |
| | trademark TEXAPON ® N70 (BASF) | |
| | NaOH (18% aqueous solution; VWR) | 0.9 |
| (c) | Mixing (a) and (b) Sub-total | 76.8 |
| (d) | APTAC-Acm copolymer sold under the trademark | 2.5 |
| | N-HANCE ® SP-100 (10% aqueous solution; | |
| | Ashland) | |
| | Water | 3.12 |
| | Microcapsule Slurry A | 0.78 |
| | Cocamidopropyl betaine sold under the | 7.9 |
| | trademark TEGO ® Betaine F50 (Evonik) | |
| | Dimethiconol & TEA-Dodecylbenzenesulfonate | 2.0 |
| | sold under the trademark BELSIL ® DM 60 10 | |
| | (Wacker) | |
| | Methylchloroisothiazolinone and | 0.1 |
| | Methylisothiazolinone (Kathon ™ CG; Brenntag) | |
| | Citric acid (10% aqueous solution; VWR) | 3.1 |
| | Water | 3.7 |
| | Total | 100 |

The deposition efficiency of Shampoo Compositions 1 and 6-9 were evaluated using the method described above. Unexpectedly, Shampoo Compositions 6-9 each had a deposition efficiency at least 4.5-fold that of Shampoo Composition 1.

### Example 3: Improved Capsule Deposition in a Shower Gel Base

A shower gel of this invention was prepared by the steps of: (a) adding 1.37 g of APTAC-Acm copolymer sold under the trademark N-HANCE® SP-100 (10% aqueous solution; Ashland) to 91.5 g of a shower gel base with constant stirring; (b) mixing 3.93 g of water and 3.2 g of Microcapsule Slurry A; (c) adding the resultant microcapsule slurry in Step (b) to the resultant shower gel base in Step (a) to obtain a shower gel composition, i.e., Shower Gel Composition 10.

A comparative shower gel was also prepared following the same procedure described above as for Shower Gel Composition 10 except that no APTAC-Acm copolymer was added.

Shower Gel Composition 10 and the Comparative Shower Gel were evaluated in a shower application. Shower Gel Composition 10 showed a fragrance intensity of 5.63 at 4 hours after application of the shower gel. By comparison, the Comparative Shower Gel had a fragrance intensity of only 5 at 4 hours after application.

### Example 4: Improved Capsule Deposition in a Bar Soap Base

A bar soap of this invention was prepared by the steps of: (a) adding 1.36 g of APTAC-Acm copolymer sold under the trademark N-HANCE® SP-100 (10% aqueous solution; Ashland) directly to 94.79 g of a bar soap base at the beginning of the finishing process (soap pellets); (b) homogenizing the soap to obtain soap noodles; and (c) adding 0.7 g water and 3.16 g Microcapsule Slurry A to obtain Bar Soap Composition 11.

A comparative bar soap was also prepared following the same procedure described above as for Bar Soap Composition 11 that no APTAC-Acm copolymer was added.

Bar Soap Composition 11 and the Comparative Bar Soap were evaluated in a hand washing application. Bar Soap Composition 11 showed a fragrance intensity of 3.3 at 2 hours after hand washing with the bar soap. By comparison, the Comparative Bar Soap had a fragrance intensity of only 1.4 at 2 hours after hand washing.

### Example 5: Comparison of APTAC-Acm and MAPTAC-Acm Copolymers

The deposition of various encapsulation technologies in a shampoo base was assessed in the presence and absence of either APTAC-Acm copolymer or methacrylamidopropyltrimethyl ammonium chloride/acrylamide (MAPTAC-Acm, supplied by SNF S.A., France) copolymer as deposition aid. Other suitable MAPTAC-Acm copolymers are described in EP 0522334 B1 and include those having a molecular weight of 500,000 Da to 30,000,000 Da, or 5,000,000 Da to 30,000,000 Da. In addition, suitable copolymers may have up to 5 mole % charged units, or alternatively more than 5 mole % charged units. Moreover, molar ratios of each monomer may be in the range of 1% to 99%. See also WO 2011002475 A1. In particular, fragrance was encapsulated in the wall polymer materials provided in Table 8.

TABLE 8

| Encapsulation | |
|---|---|
| Technology | Wall Polymer Material |
| B | Polyisocyanate sold, under the trademark |
| | TAKENATE ® D-110N (prepared in benzyl |
| | benzoate solution together with PVP/PQ11) |
| C | Polyisocyanate sold under the trademark |
| | TAKENATE ® D-110N (prepared in benzyl |
| | benzoate solution together with |
| | CMC/sulfonated polystyrene) |
| D | Polyisocyanate sold under the trademark |
| | TAKENATE ® D-110N (in ethyl acetate) |
| E | Polyacrylate |

Shampoo compositions of this invention were prepared, wherein either 0.25% APTAC-Acm copolymer or 0.25% MAPTAC-Acm copolymer in combination with Encapsulation Technologies B and C were incorporated into an unperfumed shampoo base and evaluated in a hair washing application.

Hair Swatch Preparation.

A hair swatch (natural hair swatch, 8 grams) was immersed in water (25° C., flow rate of 2 L/min) for 1 minute and subsequently squeezed to remove excess water. The hair swatch, in a Petri dish, was placed on a balance and 1 g of shampoo composition was applied onto the hair swatch. The hair swatch was lathered between the palms using 10 clockwise followed by 10 counterclockwise circular motions. The hair of the swatch was detangled and placed on a Petri dish for 15 seconds to allow the shampoo composition to be absorbed by the hair. The hair swatch was subsequently rinsed in both hands by placing the hair horizontally under a stream of water (25° C., flow rate of 2 L/min) and slowly moving the swatch side to side for 20 seconds. Excess water was gently squeezed out using two fingers.

Deposition Analysis.

After treatment (washing) of the hair swatches, wet swatches were stored in a 40 mL glass jar. Fragrance on the hair was extracted using 19.8 mL of methanol. Methyl decanoate was added as an internal standard (200 μL @ 1000 mg/L MeOH to provide a 10 mg/L in final solution). The sample was sonicated for 30 minutes, filtered with an Uptidisc filter, and liquid was recovered into a 2 mL gas chromatography (GC) vial. Samples were analyzed and quantified with GC-MS (using calibration curves). The results of this analysis are presented in Table 9.

TABLE 9

| | Deposition @ 0.25% NOE Encapsulate | | |
|---|---|---|---|
| | | With 0.25% | With 0.25% |
| Encapsulation | No Deposition | APTAC-Acm | MAPTAC-Acm |
| Technology | Aid | copolymer | copolymer |
| C | 0.9 | 11 | 19 |
| B | 0.6 | 0.9 | 2.1 |

Performance Evaluation Pre-Brush.

Panelists were instructed to lift the hair swatch by a clip, bring the sample to the nose, smell the hair in several different places of the hair swatch, evaluate the sample for overall fragrance intensity, and score the global intensity on a 0-10 scale (0=no odor, 10=very strong odor).

Performance Evaluation Post-Brush.

Panelists were instructed to brush the hair swatch once, bring the sample to the nose, smell the hair in several different places of the hair swatch, evaluate the sample for overall fragrance intensity, and score the global intensity on a 0-10 scale (0=no odor, 10=very strong odor).

The performance data on dry hair swatches are presented in Table 10.

TABLE 10

| | Intensity on Swatches @ 0.25% NOE Encaps | | | |
|---|---|---|---|---|
| | | | with 0.25% MAPTAC- | |
| Encapsulation | No Deposition Aid | | Acm copolymer | |
| Technology | Pre-Brush | Post-Brush | Pre-Brush | Post-Brush |
| C | 1 | 2 | 4 | 10 |
| E | 0 | 0.5 | 1 | 2 |

The results of these analyses indicate that the MAPTAC-Acm copolymer was particularly effective in enhancing the deposition and performance of Encapsulation Technology C and Encapsulation Technology B.

Accordingly, like the APTAC-Acm copolymer, MAPTAC-Acm copolymer can be used in combination with encapsulated fragrance compositions in finished products to enhance the deposition of the fragrance onto surfaces. The MAPTAC-Acm copolymer maybe present in the final product in an amount in the range of 0.05 wt % to 0.25 wt % of the final product.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Indeed, to achieve the purpose of encapsulating an active material, one skilled in the art can design and prepare a capsule composition by using different encapsulating polymers, coatings, polyfunctional nucleophiles and/or electrophiles, and/or capsule formation aids, varying the concentrations of these wall-forming materials and/or catalysts to achieve desirable organoleptic or release profiles in a consumable product. Further, the ratios among polyfunctional nucleophiles and/or electrophiles, capsule forming aids, adjuvants, core modifiers, active materials, and catalysts can also be determined by a skilled artisan through assays known in the art to prepare capsule compositions with desirable properties.

From the above description, a skilled artisan can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A microcapsule composition comprising a microcapsule and a copolymer of acrylamidopropyl trimonium chloride and acrylamide as a first deposition aid to facilitate the deposition of the microcapsule onto a hard surface, wherein
the microcapsule having an oil core and a microcapsule wall encapsulating the oil core,
the microcapsule oil core contains a fragrance,
the microcapsule wall is formed of an encapsulating polymer,
the encapsulating polymer contains the reaction product of a multi-functional electrophile and a multi-functional nucleophile,
the multi-functional electrophile contains a polyisocyanate, and
the multi-functional nucleophile contains a branched polyethylene imine.

2. The microcapsule composition of claim 1, wherein the microcapsule composition contains by weight 0.1 wt % to 90 wt % of the microcapsule and 0.01 wt % to 50 wt % of the copolymer of acrylamidopropyl trimonium chloride and acrylamide.

3. The microcapsule composition of claim 1, wherein the microcapsule has a zeta potential of 25 mV or greater and a rupture force of 5 mN or less.

4. The microcapsule composition of claim 1, wherein the microcapsule has a size of 0.1 micron to 1000 microns; the branched polyethyleneimine has a molecular weight of 750 Da to 50000 Da; and the polyisocyanate is a trimer of hexamethylene diisocyanate, a trimer of isophorone diisocyanate, a biuret of hexamethylene diisocyanate, a polyisocyanurate of toluene diisocyanate, a trimethylol propane-adduct of toluene diisocyanate, a trimethylol propane-adduct of xylylene diisocyanate, or a combination thereof.

5. The microcapsule composition of claim 1, wherein the multi-functional nucleophile is a mixture of a branched polyethyleneimine and a polyamine selected from the group consisting of hexamethylenediamine, ethylenediamine, 1,3-diaminopropane, 1,4-diaminobutane, diethylenetriamine, pentaethylenehexamine, bis(3-aminopropyl)amine, bis(hexanethylene)triamine, tris(2-aminoethyl)amine, triethylenetetramine, N,N′-bis(3-aminopropyl)-1,3-propanediamine, tetraethylenepentamine, pentaethylenehexamine, chitosan, nisin, gelatin, 1,3-diamino-guanidine, 1,1-dimethylbiguanide, guanidine, arginine, lysine, ornithine, and combinations thereof.

6. The microcapsule composition of claim 1, further comprising a second, third, fourth, fifth, or sixth delivery system.

7. The microcapsule composition of claim 1, further comprising a second deposition aid selected from the group consisting of polyquaternium-4, polyquaternium-5, polyquaternium-6, polyquaternium-7, polyquaternium-10, polyquaternium-16, polyquaternium-22, polyquaternium-24, polyquaternium-28, polyquaternium-39, polyquaternium-44, polyquaternium-46, polyquaternium-47, polyquaternium-53, polyquaternium-55, polyquaternium-67, polyquaternium-68, polyquaternium-69, polyquaternium-73, polyquaternium-74, polyquaternium-77, polyquaternium-78, polyquaternium-79, polyquaternium-80, polyquaternium-81, polyquaternium-82, polyquaternium-86, polyquaternium-88, polyquaternium-101, polyvinylamine, polyethyleneimine, polyvinylamine and vinylformamide copolymer, and combinations thereof.

8. The microcapsule composition of claim 1, further comprising a capsule formation aid selected from the group consisting of a polyvinyl alcohol, polystyrene sulfonate, carboxymethyl cellulose, naphthalene sulfonate condensate salt, polyvinylpyrrolidone, copolymer of vinyl pyrrolidone and quaternized dimethylaminoethyl methacrylate, and combinations thereof.

9. A consumer product comprising the microcapsule composition of claim 1.

10. The consumer product of claim 9, wherein the consumer product is a hair care product, a personal care product, a fabric care product, or a home care product.

11. The consumer product of claim 9, wherein the consumer product is a shampoo, shower gel, or bar soap.

12. A method of preparing a shampoo composition comprising the step of:
(i) providing a shampoo base containing 2 wt % to 35 wt % of a surfactant, and
(ii) adding into the shampoo base a microcapsule and a copolymer of acrylamidopropyl trimonium chloride and acrylamide to obtain the shampoo composition,
wherein the shampoo composition contains by weight (a) 0.001% to 10% of the copolymer of acrylamide and acrylamidopropyltrimonium chloride, and (b) 0.01% to 10% of the microcapsule, the microcapsule contains a microcapsule core and a microcapsule wall encapsulating the microcapsule core, the microcapsule core contains a fragrance, and the microcapsule wall is formed of an encapsulating polymer.

13. The method of claim 12, wherein the microcapsule is prepared by the steps of:
(iii) preparing an oil phase containing a fragrance and at least one polyisocyanate;
(iv) preparing an aqueous phase containing a microcapsule formation aid;
(v) emulsifying the oil phase into the aqueous phase to form a fragrance emulsion;
(vi) adding at least one cross-linking agent to the fragrance emulsion to form a polyurea capsule slurry; and
(vii) curing the polyurea capsule slurry.

14. The method of claim 13, wherein the polyisocyanate contains a trimethylol propane-adduct of xylylene diisocyanate and is present in a polyisocyanate solution having a flash point of at least 60° C.
